(19) United States
(12) Patent Application Publication
(10) Pub. No.: US 2025/0339203 A1
(43) Pub. Date: Nov. 6, 2025

(54) PLANNING AN INTRAORAL TREATMENT

(71) Applicant: EXOCAD GMBH, Darmstadt (DE)

(72) Inventor: Gustavo ISMAEL, Sao Paulo (BR)

(21) Appl. No.: 18/656,374

(22) Filed: May 6, 2024

(51) Int. Cl. A61B 34/10; A61C 13/34; G16H 20/40

(52) U.S. Cl. CPC A61B 34/10; A61C 13/34; G16H 20/40; A61B 2034/102

(57) ABSTRACT

The invention relates to a method for planning an intraoral treatment for a patient. A three-dimensional digital tissue model of intraoral tissue of the patient's oral cavity comprising maxillary and mandibular tissue is received. A maximum mouth opening value is received. Relative positions of the maxillary and mandibular tissue relative to each other are determined for a maximum mouth opening of the patient using the maximum mouth opening value. Size information of one or more treatment elements selected to be used for executing the intraoral treatment are received. A three-dimensional working space required for arranging and using the one or more selected treatment elements for executing the intraoral treatment is determined using the size information of the one or more selected treatment elements.

### FIELD OF THE INVENTION

The invention relates to the field of dental technology, in particular to a method for planning an intraoral treatment for a patient.

### BACKGROUND

Different patients may be able to open their mouths up to different degrees. Such different degrees of mouth opening achievable by different patient may pose a challenge for intraoral treatments, like, e.g., a dental implant static-guided surgery. Patients with open mouth limitations may provide only a limited access to a treatment site within their oral cavity, making an accurate treatment difficult. Due to a mouth opening limitation there may be an insufficient free space within a patient's oral cavity for using pre-selected tools to execute a treatment. As a result, execution of the treatment may be challenging. The treatment may be executed less accurately compared to a case of a larger degree of mouth opening or the treatment may even have to be aborted, due to complications arising from the mouth opening limitation.

### SUMMARY

It is an objective to provide for a method for planning an intraoral treatment for a patient, a computer program product for planning an intraoral treatment for a patient, and a computer device for planning of an intraoral treatment for a patient.

In one aspect, the invention relates to a method for planning an intraoral treatment for a patient. The method comprises receiving a three-dimensional digital tissue model of intraoral tissue of the patient's oral cavity comprising maxillary and mandibular tissue. Further, a maximum mouth opening value descriptive of a maximum degree of mouth opening achievable by the patient is received. Relative positions of the maxillary and mandibular tissue of the three-dimensional digital tissue model relative to each other are determined for a maximum mouth opening of the patient using the maximum mouth opening value. Size information descriptive of sizes of one or more treatment elements selected to be used for executing the intraoral treatment are received. A three-dimensional working space required for arranging and using the one or more selected treatment elements for executing the intraoral treatment is determined using the size information of the one or more selected treatment elements.

For a test position and test orientation of the one or more selected treatment elements within the three-dimensional digital tissue model with the maxillary and mandibular tissue arranged at the determined relative positions it is checked, whether the working space arranged at the test position and aligned with the test orientation intersects with tissue of the three-dimensional digital tissue model opposite of the test position. In response to a detection of an intersection, an indication signal indicating the detected intersection is output and a planning adjustment for the planning of the intraoral treatment configured to prevent the detected intersection is determined.

For intraoral treatments a maximum degree of mouth opening achievable by a patient may be a limiting factor. For example, for patients with open-mouth limitations some treatment elements selected and/or required for the intraoral treatment may not be usable due to restrictions arising from the open-mouth limitations, i.e., limitations of the maximum degree of mouth openings achievable by the patients.

For example, for dental implant static-guided surgery a maximum degree of mouth opening achievable by a patient may be a crucial limiting factor. Static-guided implant surgery, e.g., relies heavily on specialized treatment elements in form of specialized treatment tools. These treatment tools, like, e.g., implant contra-angle, implant drill, drill guide handle, sleeve, and/or surgery guide, enable an accurate placing of implants.

However, in case of limitations of the maximum degree of mouth opening achievable, an intraoral treatment may become challenging. Patients with open mouth limitations may provide a limited access to a treatment site within the patient's oral cavity, e.g., a surgical site. Such a limited access may make it difficult to place, e.g., an implant accurately. A limited working space, in particular a working space of insufficient size may interfere with the final implant position resulting in a less accurate result of the treatment, e.g., a less accurate surgery, or may even require an abortion of the intraoral treatment, e.g., a surgery.

Examples may have the beneficial effect that a maximum degree of mouth opening of a patient and its impact on the intraoral treatment, in particular on the treatment elements to be used for the intraoral treatment are already taken into account during planning of the dental treatment. Thus, problems arising during an intraoral treatment from limitations of the working space available for the intraoral treatment due to mouth opening limitations of a patient may be avoided. In particular, inaccurate results of the intraoral treatment or even an abortion of the intraoral treatment due to the limitations may be prevented.

Taking into account a maximum degree of mouth opening achievable by a patient as well as sizes of treatment elements selected to be used for executing the intraoral treatment for the planning of the intraoral treatment may avoid problem arising from a lack of an insufficiently large working space within the oral cavity of the patient.

The three-dimensional digital tissue model is a three-dimensional digital of intraoral tissue of the patient's oral cavity. It may provide information about surfaces structures of the patient's intraoral tissue. It may, e.g., further provide information about internal structures of the patient's intraoral tissue. The intraoral tissue comprises maxillary and mandibular tissue. The three-dimensional digital tissue model may, e.g., comprise a first three-dimensional digital model of maxillary tissue and a second three-dimensional digital model of mandibular tissue. The first and the second three-dimensional digital model may be positionable independently of each other.

For example, the intraoral tissue comprises hard tissue, like, e.g., teeth or bone tissue. For example, the intraoral tissue comprises soft tissue, like, e.g., gingiva tissue. For example, maxillary tissue comprised by the intraoral tissue comprises maxillary hard tissue, like, e.g., maxillary teeth or maxillary bone tissue. For example, mandibular tissue comprised by the intraoral tissue comprises mandibular hard tissue, like, e.g., mandibular teeth or mandibular bone tissue.

The three-dimensional digital tissue model of intraoral tissue may, e.g., comprise scan data of the intraoral tissue. The scan data of the intraoral tissue may, e.g., comprise scan data acquired using a medical imaging technique, like computed tomography (CT), cone beam computed tomography (CBCT), and/or digital volume tomography (DVT). The scan data of the intraoral tissue may comprise, e.g., optical scan data. The optical scan data may, e.g., comprise intraoral optical scan data or optical scan data from an optical scan of a classical mold/impression of the intraoral tissue. The optical scan data may, e.g., provide information about the surface structure of the patient's intraoral tissue comprising teeth and the gingiva.

For example, the maximum mouth opening value is a value determined using a maximal interincisal opening (MIO) measurement or a range of motion (ROM) measurement.

MIO measures the distance between the central incisors, when a patient's mouth is fully open. Normal mouth-opening ranges from 35 mm to 45 mm. Males usually have slightly greater mouth opening than females, i.e. 40 mm to 60 mm with an average of about 50 mm. ROM is the maximum distance the mandible moves. Sometimes ROM and MIO may be different, e.g., in case of a patient with an open bite. If a MIO is less than 35 mm, it is referred to as trismus. Trismus is a condition of restricted opening of the mouth. It may interfere with eating, speaking, and maintaining proper oral hygiene. Furthermore, trismus may interfere with intraoral treatments by a dentist.

To determine a patient's MIO, a special scale may be used comprising at least one range for measuring how wide a patient can open the mouth. Such a scale may, e.g., show different ranges of how wide a patient can open the mouth. These different ranges may, e.g., including a capacity for trismus and a smaller range for people who have even more trouble opening their mouth.

The maximum mouth opening value may quantify the maximum degree of mouth opening achievable by the patient, e.g., in degrees [°] or millimeters [mm]. In case the maximum mouth opening value is quantified in millimeters [mm], it may describe a maximum distance achievable between the maxillary and mandibular intraoral tissue, e.g., a maximum distance between central incisors. In case the maximum mouth opening value is quantified in degrees [°], it may describe a maximum opening angle achievable between the maxillary and mandibular intraoral tissue, e.g., a maximum distance between central incisors. The angle may be determined relative to an axis of rotation, e.g., relative to an axis of rotation extending through the temporomandibular joints.

This maximum degree of mouth opening achievable by the patient, which is described by the maximum mouth opening value, defines a physical boundary of jaw movement achievable by the patient.

The maximum mouth opening value may, e.g., be a patient-specific value describing an individual maximum degree of mouth opening achievable by the individual patient, for which the intraoral treatment is planned. For example, the maximum mouth opening value may be a value measured for the individual patient. For example, the maximum mouth opening value may, e.g., be an average value descriptive of a maximum degree of mouth opening achievable by the patient based on measurements of mouth opening executed for a reference group of patients. The reference group of patients may, e.g., comprise a representative reference group of patients with physiological parameters similar to physiological parameters of the patient, for which the intraoral treatment is planned. For example, the patients of the reference group may be patients of same age, same gender, and/or with other same parameters. For the patients of the reference group, e.g., individual maximum mouth opening value may be measured and an average value determined using the measured individual values. The average value may, e.g., be used as the maximum mouth opening value descriptive of the maximum degree of mouth opening achievable by the patient, for which the intraoral treatment is planned, if a patient-specific value is not available.

The maximum mouth opening value may be used for determining relative positions of the maxillary and mandibular tissue of the three-dimensional digital tissue model relative to each other for a situation, in which the patient opens the mouth to a maximal possible extend. The three-dimensional digital model of the maxillary tissue and the three-dimensional digital model of the mandibular tissue of the three-dimensional digital tissue model may be positioned relative to each other in these determined relative positions.

For example, a digital articulator may be used to determine the relative positions of the maxillary and mandibular tissue according to the maximum mouth opening value. The digital articulator may be configured to simulate movements of the mandible in relation to the maxilla. Thus, using the digital articulator the maxillary and mandibular tissue may be positioned in anatomically correct positions relative to each other. Thus, it may not only be ensured that a distance between the maxillary and mandibular tissue corresponds to the maximum mouth opening value, but that positions of the maxillary and mandibular tissue are also anatomically correct. For example, jaw movements of the patient, i.e., movements of the mandible relative to the maxilla, may be tracked and used for determining the relative positions of the maxillary and mandibular tissue according to the maximum mouth opening value. For example, jaw motion tracking data may be acquired, which comprises the maximum mouth opening value. Thus, the maximum mouth opening value may, e.g., be received as part of the jaw motion tracking data. For example, the digital articulator may be configured to use jaw motion tracking data for determine the relative positions of the maxillary and mandibular tissue according to the maximum mouth opening value.

Using jaw motion tracking data, not only a maximum mouth opening may be taken into account, but also the openings achievable by the jaws in all directions may be taken into account as well as movements of the jaws in all possible directions. For acquiring the jaw motion tracking data, e.g., an electronic jaw registration and movement tracking system may be used. In case no jaw motion tracking data acquired for the individual patient are available, e.g., averaged values for the jaw motions may be used. This jaw motion tracking data describing possible jaw motions may define further physical boundaries of jaw movement achievable by the patient.

For example, an articulator-free movement virtualization may be used to determine the relative positions of the maxillary and mandibular tissue according to the maximum mouth opening value. For the articulator-free movement virtualization, e.g., jaw motion tracking data may be used. In case no jaw motion tracking data acquired for the individual patient are available, e.g., averaged values for the jaw motions may be used.

In addition, size information descriptive of sizes of one or more treatment elements selected to be used for executing the intraoral treatment are provided. The size information may, e.g., comprise parameters descriptive of the sizes of the one or more treatment elements. The size information may, e.g., be provided using one or more three-dimensional digital models of the one or more treatment elements selected to be used for executing the intraoral treatment are provided. For example, a library may be provided comprising parameters descriptive of sizes of a plurality of different treatment elements and/or comprising a plurality of three-dimensional digital models of a plurality of different treatment elements. The one or more treatment elements to be used for executing the intraoral treatment may, e.g., be selected using the respective library.

A three-dimensional working space required for arranging and using the one or more selected treatment elements for executing the intraoral treatment is determined using the size information of the one or more selected treatment elements. For example, the sizes of the intraoral treatment are combined, in order to determine a size of the three-dimensional working space required for arranging and using the one or more selected treatment elements.

In case different combinations of treatment elements are used for different steps of the intraoral treatment, it is e.g., determined which combination of treatment elements is required for which step of the intraoral treatment. For each of these combinations a three-dimensional working space may be determined, which is required to arrange and use the respective combination of treatment elements within the oral cavity of the patient.

For a test position and test orientation of the one or more selected treatment elements within the three-dimensional digital tissue model with the maxillary and mandibular tissue arranged at the determined relative positions it is checked, whether the working space arranged at the test position and aligned with the test orientation intersects with tissue of the three-dimensional digital tissue model opposite of the test position. Thus, e.g., a virtual safe space may be defined in form of the three-dimensional working space. For this movement tracking it may, e.g., be determined, whether collisions may occur between the selected treatment elements and with tissue of the antagonist jaw. In case an intersection is detected, there a collision between the selected treatment elements and with tissue of the antagonist jaw may occur. The test position and test orientation of the one or more selected treatment elements may result from a preliminary position and orientation determined for an implant to be inserted.

Opposite of the test position may, e.g., refer to opposite in a direction parallel to the test orientation.

In response to a detection of an intersection, an indication signal is output indicating the detected intersection. Furthermore, a planning adjustment for the planning of the intraoral treatment configured to prevent the detected intersection.

The planning adjustment may, e.g., comprise an adjusting of the test position and/or the test orientation resulting in an adjusted position and/or an adjusted orientation, for which an intersection of the three-dimensional workspace with tissue of the three-dimensional digital tissue model opposite of the adjusted position is prevented. The planning adjustment may, e.g., comprise determining one or more replacement treatment elements as replacements for one or more of the planned treatment elements. These replacement treatment elements may have sizes resulting in an adjusted three-dimensional workspace, e.g., a smaller three-dimensional workspace, for which at the test position with the test orientation an intersection with tissue of the three-dimensional digital tissue model opposite of the test orientation is prevented. For example, the replacing of the test position, of the test orientation and/or of one or more of the planned treatment elements may be combined. In this case, the adjusted three-dimensional workspace may be configured such that at the adjusted position and/or with the adjusted orientation an intersection of the adjusted three-dimensional workspace with tissue of the three-dimensional digital tissue model opposite of the test orientation is prevented.

The planning the interporal treatment may, e.g., comprise planning one or more implants. The determining of the three-dimensional working space may, e.g., be part of an implant planning, i.e., a planning of positions of implants to be inserted into the intraoral tissue of the patient.

For example, the method further comprises receiving a clearance value defining a size of an additional clearance required for a moving and positioning of the one or more selected treatment elements at the test position with the test orientation. The clearance value is used for determining the three-dimensional working space.

Examples may have the beneficial effect that for determining the three-dimensional working space not only the sizes of the selected treatment elements are taken into account, but also a clearance. The size of the selected treatment elements may, e.g., be used to define a preliminary three-dimensional working space, to which the additional clearance is added to determine the final three-dimensional working space to be used for the checking for the test position and test orientation.

The form of the three-dimensional working space may, e.g., be given by the form of the selected treatment elements. The form of the three-dimensional working space may, e.g., be or comprise a simplified geometrical form. The form of the three-dimensional working space may, e.g., be or comprise a rotationally symmetric form, like, e.g., a cylinder or a conical frustum. The size of the three-dimensional working space may, e.g., be selected such that it envelops the selected treatment elements.

The additional clearance may, e.g., be added to a preliminary three-dimensional working space given by the form of the selected treatment elements. The additional clearance may, e.g., be added to a preliminary three-dimensional working space given by a simplified geometrical form as described above. The resulting final three-dimensional working space may, e.g., have the form of the selected treatment elements. The resulting final three-dimensional working space may, e.g., have a simplified geometrical form as described above.

For example, the method further comprises using a digital articulator for determining the relative positions of the maxillary and mandibular tissue relative to each other.

An articulator is a mechanical hinged device, which is used to reproduce some or all the movements of a mandible in relation to a maxilla. The articulator is configured to simulate the position and movements of the bilateral temporomandibular joints, which determine the relative movements of mandible and maxilla. The digital articulator may, e.g., be configured to mimic individual movements of the maxillary and mandibular tissue relative to each other defined by tracking information, which is acquired by tracking movements of a patient's jaws. The digital articulator may, e.g., be configured to mimic movements of the maxillary and mandibular tissue defined by averaged moving information of human jaw movements.

A model of a patient's maxillary and mandibular tissue is positioned within the articulator and their relative positions and movements may be simulated using the articulator. A digital articulator simulates the movements of a physical articulator. The three-dimensional digital tissue model comprising the maxillary and mandibular tissue, e.g., in form of a three-dimensional digital maxillary tissue model and a three-dimensional digital mandibular tissue model, may be arranged within the digital articulator. The relative position between the maxillary and mandibular tissue within the digital articulator may, e.g., be determined using a measurement of a relative position of the patient's maxillary and mandibular tissue in the oral cavity. For this measurement, e.g., a facebow may be used. The digital articulator may then, e.g., be opened with the maxillary and mandibular tissue arranged therein simulating an opening of the patient's mouth. The digital articulator may, e.g., be opened until the maximum degree of mouth opening described maximum mouth opening value is reached. Thus, anatomically correct relative positions of the maxillary and mandibular tissue of the three-dimensional digital tissue model relative to each other may be determined for the maximum mouth opening of the patient according to the maximum mouth opening value.

For example, the digital articulator may comprise a three-dimensional digital model of a physical articulator configured to simulate the settings and movements of the physical articulator.

A facebow is a device, which is used to measure the position of the maxilla of the patient relative to the mandible and/or the temporomandibular joints, which are simulated by the articulator. For a digital articulator an electronic facebow may be used, which is configured to acquire and provide digital data descriptive of the respective position of the patient's maxilla relative to the mandible and/or temporomandibular joints. Such an electronic facebow, sometimes also referred to as a digital facebow, may measures parameters regarding the position of the patient's jaws and provide the measured data in digital form. The virtual position data of the patient's jaws may be transferred computer and imported into a program providing the digital articulator. The electronic facebow may for example be used to determine the position data of the maxilla in relation to the base of the skull and/or the temporomandibular joints. The electronic facebow may, e.g., use the patient's external auditory canals and nose as reference positions. For example, the electronic facebow may, e.g., be fixed on both sides of the external auditory canals, e.g., with olives, and with a nose support on the patient's head. Alternatively, the electronic facebow may, e.g., be fixed to the patient's head and the relative position of the external auditory canals and/or the nose may be measured. Furthermore, the position of the maxilla and/or the mandible relative to the reference positions may, e.g., be determined. For example, a mouthpiece, like a bite fork, may be provided. The mouthpiece is, e.g., pressed against the chewing surfaces or incisal edges of the maxillary teeth to measure their position. Furthermore, the position data of the mandible, in relation to the maxilla may be measured. Thus, a registration of the position(s) of the patient's jaw(s) may be provided.

For the determining of the relative positions of the maxillary and mandibular tissue relative to each other using the digital articulator, the method, e.g., further comprises receiving tracking information of a tracking of jaw movements of the patient's and using the received tracking information for defining a movement of the maxillary and mandibular tissue relative to each other with the digital articulator.

A simulation of an opening of the patient's mouth may, e.g., be executed using data comprising tracking information provided by a jaw movement registration and tracking system. The digital articulator may, e.g., be configured to mimic movements of the maxillary and mandibular tissue relative to each other defined by the tracking information, which are acquired by tracking movements of the patient's jaw. Thus, the digital articulator may, e.g., take into account patient individual features of jaw movement, when determining of the relative positions of the maxillary and mandibular tissue. When simulating an opening of the mouth, the digital articulator may, e.g., mimic a patient individual jaw movement as defined by the tracking information. The digital articulator may, e.g., be opened mimicking the patient individual jaw movement until the maximum degree of mouth opening described maximum mouth opening value is reached. Thus, an individually anatomically correct relative positions of the maxillary and mandibular tissue of the three-dimensional digital tissue model relative to each may be determined for the maximum mouth opening of the patient according to the maximum mouth opening value. For example, the maximum mouth opening value may be received as part of the tracking information describing of a maximum degree of mouth opening achievable by the patient.

The jaw movement registration and tracking system may, e.g., comprise an electronic facebow, which is used to register and track movements of the patient's jaws. The jaw movement registration and tracking system may, e.g., be configured for register and track movements of the patient's jaws using a motion capture method. The motion capture method may, e.g., use marker, like magnets, for capturing the motion. The motion capture method may, e.g., use a markerless approach, e.g., with one or more optical sensors configured for optically capturing jaw movements.

For the determining of the relative positions of the maxillary and mandibular tissue relative to each other using the digital articulator, the method, e.g., further comprises receiving averaged moving information of human jaw movements and using the received moving information for defining the movement of the maxillary and mandibular tissue relative to each other with the digital articulator.

The digital articulator may, e.g., be configured to simulate movements of the maxillary and mandibular tissue relative to each other corresponding to averaged human jaw movements. For this purpose, averaged moving information of human jaw movements may be used. This averaged moving information may, e.g., be acquired by tracking jaw movements of a plurality of patient's and averaging the resulting tracking information. Thus, the digital articulator may, e.g., take into account averaged human jaw movements, when determining of the relative positions of the maxillary and mandibular tissue. When simulating an opening of the mouth, the digital articulator may, e.g., simulate averaged human jaw movements as defined by the averaged moving information. The digital articulator may, e.g., be opened mimicking an averaged human jaw movement until the maximum degree of mouth opening described maximum mouth opening value is reached.

For example, the size information descriptive of the sizes of the one or more selected treatment elements are received from one or more libraries of treatment elements comprising a plurality of entries assigned to different treatment elements of a plurality of different treatment elements with individual entries of the plurality of entries comprising size information of individual treatment elements of the plurality of treatment elements, to which the individual entries are assigned.

For example, the one or more treatment elements may be selected from the one or more libraries of treatment elements and the size information comprised by the entries assigned to the selected treatment elements provided upon selection of the respective treatment elements.

For example, the one or more libraries of treatment elements may comprise three-dimensional digital models of the treatment elements. For example, the three-dimensional digital models of the selected treatment elements may be used for determining the three-dimensional working space. For example, the size information descriptive of sizes of the selected one or more treatment elements may be comprised by the three-dimensional digital models of the respective treatment elements.

For example, the one or more libraries comprise a plurality of treatment elements for the same specific task. The treatment elements may, e.g., vary regarding form, size and/or length. Thus, a treatment element may be selected with a size, such that in the test position with the test orientation an intersect of one of the selected treatment elements with tissue of the three-dimensional digital tissue model opposite of the test position can be prevented. For example, one or more alternative treatment elements may be selected, which are assigned to the same task as previously selected treatment elements, which are to be replaced by the alternative treatment elements. The one or more alternative treatment elements may be selected, such that an intersection is avoided using the alternative treatment elements. For example, the one or more alternative treatment elements may have forms, sizes and/or lengths deviating from forms, sizes and/or lengths of previously selected treatment elements, such that an intersection is avoided using the alternative treatment elements.

For example, the outputting of the indication signal comprises a highlighting of the intersection in a graphical visualization of the three-dimensional digital tissue model with the maxillary and mandibular tissue arranged in the relative positions relative to each other. The graphical visualization of the three-dimensional digital tissue model with the highlighted intersection is output using a graphical user interface.

The highlighting of the intersection may intuitively indicate, where problems from a limited mouth opening for a planned intraoral treatment may arise. Thus, the used may, e.g., decided whether to adjust the test position, the test orientation, and/or one or more of the selected one or more treatment elements, e.g., by selecting one or more alternative treatment elements for replacing one or more of the selected treatment elements. The adjusted test position may be chosen to avoid the highlighted intersection. The adjusted test orientation may be chosen to avoid the highlighted intersection. The one or more alternative treatment elements may be chosen depending on their forms, sizes and/or lengths deviating from forms, sizes and/or lengths of previously selected treatment elements, in order to the highlighted intersection.

The indication signal may, e.g., be a warning signal warning of an intersection. For example, the intersection may be highlighted in the graphical visualization of the three-dimensional digital tissue model.

For example, the method further comprises providing the planning adjustment and preventing the detected intersection. The determined planning adjustment may be applied and thereby the previously detected intersection avoided. For example, an adjustment of the test position, an adjustment of the test orientation and/or a replacement of one or more of the selected treatment elements may be applied.

For example, the test position for the one or more selected treatment elements is a position of a plurality of potential position tested for the planned treatment. The determined planning adjustment compromises a restriction of a selectability of the positions of the plurality of positions. The test position is excluded from the selectability for the planned treatment.

Example may have the beneficial effect that positions, like the test position, for which a working space intersects with tissue of the three-dimensional digital tissue model opposite of the test position, are excluded from the selectability for the planned treatment. Thus, when planning the treatment, a user may be prevented from selecting a position for the one or more selected treatment elements, at which an intersection occurs. The user may, e.g., change a position of the more selected treatment elements, e.g., by moving three-dimensional digital models of the more selected treatment elements relative to the three-dimensional digital tissue model. When a position is reached, at which an intersection would occur, the user may be prevented from moving the three-dimensional digital models of the more selected treatment elements further into the direction of the position, at which the intersection would occur.

Example may comprise automatically checking for all the positions of the plurality of potential positions tested for the planned treatment, whether an intersection of the three-dimensional working space with the tissue of the three-dimensional digital tissue model opposite of the test position occurs. Those positions of the plurality of the potential positions, for which an intersection is determined, may be excluded from the selectability for the planned treatment. The positions of the plurality of potential positions tested for the planned treatment may, e.g., comprise positions within a predefined radius around a reference test position. The reference test position may be a test position selected by the user or a test position suggested automatically, e.g., based on information descriptive of a type of treatment to be planned and elements of the intraoral tissue to be targeted by the respective treatment. In case of a planning of implants, the type of treatment may, e.g., be an inserting of implants for one or more single teeth to be restored and/or for one of more bridges to be used for restoring teeth. The information describing elements of the intraoral tissue to be targeted by the respective treatment may, e.g., identify the one or more teeth to be restored.

Thus, e.g., a selection of a position of a treatment element for a planned treatment, like a position of an implant, may be restricted, such that a position cannot be selected, if for this position an intersection is determined. For example, during an implant planning, it may not be possible to move an implant to a position, at which an intersection occurs. A movement of a three-dimensional digital model of the treatment element to the position, at which an intersection occurs, may be blocked. Thus, when approaching the position, at which an intersection occurs, the user may be prevented from moving the three-dimensional digital model of the treatment element further towards this position.

The determining of the intersection may take into account more than one treatment element, while only a graphical visualization of a single treatment element is moved around within a graphical visualization of the three-dimensional digital tissue model of intraoral tissue using a graphical user interface. For example, an implant may be moved around, while also the treatment elements required for preparing and/or inserting the implant are taken into account.

For example, the test orientation of the one or more selected treatment elements is an orientation of a plurality of potential orientation tested for the planned treatment. The determined planning adjustment compromises a restriction of a selectability of the orientations of the plurality of orientations. The test orientation is excluded from the selectability for the planned treatment.

Example may have the beneficial effect orientations, like the test orientation, for which a working space intersects with tissue of the three-dimensional digital tissue model opposite of the test position, are excluded from the selectability for the planned treatment. Thus, when planning the treatment, a user may be prevented from selecting an orientation for the one or more selected treatment elements, at which an intersection occurs. The user may, e.g., change an orientation of the more selected treatment elements, e.g., by moving three-dimensional digital models of the more selected treatment elements relative to the three-dimensional digital tissue model. When an orientation is reached, at which an intersection would occur, the user may be prevented from further moving the orientation of the three-dimensional digital models of the more selected treatment elements into the direction of the orientation, at which the intersection would occur.

Example may comprise automatically checking for all the orientations of the plurality of potential orientations tested for the planned treatment, whether an intersection of the three-dimensional working space with the tissue of the three-dimensional digital tissue model opposite of the test position occurs. Those orientations of the plurality of the potential orientations, for which an intersection is determined, may be excluded from the selectability for the planned treatment. The orientations of the plurality of potential orientations tested for the planned treatment may, e.g., comprise orientations within a predefined right circular cone around a reference test orientation. The reference test orientation coincides with an axis of the right circular cone, while the apex of the cone is arranged at the test position, for which the test orientations are considered. An angle θ between a generatrix of the cone and the axis of the cone may define a maximum deviation of the orientations of the plurality of the potential orientations taken into account from the reference test orientation. In case of a spatially extended test position, a predefined frustum, e.g., a right conical frustum, may be used to define the plurality of potential orientations tested for the planned treatment. For example, the smaller basis of the predefined frustum may be provided by the spatially extended test position or may comprise the spatially extended test position.

The reference test orientation may be a test orientation selected by the user or a test orientation suggested automatically, e.g., based on information descriptive of a type of treatment to be planned and elements of the intraoral tissue to be targeted by the respective treatment. In case of a planning of implants, the type of treatment may, e.g., be an inserting of implants for one or more single teeth to be restored and/or for one of more bridges to be used for restoring teeth. The information describing elements of the intraoral tissue to be targeted by the respective treatment may, e.g., identify the one or more teeth to be restored.

Thus, e.g., a selection of an orientation of a treatment element for a planned treatment, like an orientation of an implant, may be restricted, such that an orientation cannot be selected, if for this orientation an intersection is determined. For example, during an implant planning, it may not be possible to change an orientation of an implant to a position, at which an intersection occurs. An alignment of a three-dimensional digital model of the treatment element with an orientation, for which an intersection occurs, may be blocked. Thus, when approaching an orientation, at which an intersection occurs, the user may be prevented from rotating the three-dimensional digital model of the treatment element further towards this orientation.

The determining of the intersection may take into account more than one treatment element, while only a graphical visualization of a single treatment element is oriented within a graphical visualization of the three-dimensional digital tissue model of intraoral tissue using a graphical user interface. For example, an alignment of an implant may be change, while also the treatment elements required for preparing and/or inserting the implant are taken into account.

For example, the plurality of potential position tested for the planned treatment and the plurality of potential orientations tested for the planned treatment may both be taken into account. For example, for each of the test positions of the plurality of potential position tested the plurality of potential orientations may be taken into account.

For example, the method further comprises outputting a graphical visualization of the treatment elements using the graphical user interface. The determined planning adjustment comprises a restriction of a movability of the treatment elements relative to the three-dimensional digital tissue model. One or more of the following is excluded: a moving of the treatment elements to the test position, a moving of the treatment elements into alignment with the test orientation.

For outputting the graphical visualization of the treatment elements, e.g., three-dimensional digital models of the treatment elements may be used and graphically displayed. The treatment elements may be graphically displayed together with the three-dimensional digital tissue model. When planning the treatment, in particular a positioning and alignment of the treatment elements used for the treatment, the treatment elements may be moved relative to the three-dimensional digital tissue model. The moving of the treatment elements may comprise a moving of the treatment elements to the test position and/or a moving of the treatment elements into alignment with the test orientation, i.e., an adjusting of the orientation of the treatment elements. This moving of the treatment elements may be restricted. Consequently, a moving of the treatment elements to the test position, for which an intersection occurs, and/or a moving of the treatment elements into alignment with the test orientation, for which an intersection occurs, may be excluded by the restriction.

For example, the determining of the planning adjustment comprises determining an adjusted position as a replacement for the test position, at which an intersection of the three-dimensional workspace with tissue of the three-dimensional digital tissue model opposite of the adjusted position is prevented. The adjusted position is output.

Examples may have the beneficial effect that a recommendation for a replacement for the test position in form of the adjusted position is output. At this adjusted position no intersection of the three-dimensional workspace with tissue of the three-dimensional digital tissue model opposite of the adjusted position occurs. The determining of the adjusted position may, e.g., comprise checking alternative positions in the vicinity of the test position, e.g., within a predefined radius around the test position. As adjusted position, e.g., a nearest alternative position is being selected, at which no intersection occurs. In case no alternative position without intersection can be determined within the predefined radius, the radius may, e.g., successively be extended, until one or more alternative positions without intersection are found.

In case the test position is the result of a moving of the replacement elements, i.e., is reached by the respective movement, the determining of the adjusted position may, e.g., comprise moving further, until a position is reach, at which no intersection occurs. The moving further may follow a previous direction of movement. For example, the moving further may follow a direction extrapolated from the previous moving, i.e., from a trajectory of the previous moving.

In case the test position is the result of a moving of the replacement elements, i.e., is reached by the respective movement, the determining of the adjusted position may, e.g., comprise moving back, until a position is reach, at which no intersection occurs. The moving back may, e.g., follow a trajectory of the previous moving, until a position is reach, at which no intersection occurs.

For example, the adjusted position may be proposed or automatically executed, i.e., the test position may be automatically replaced by the adjusted position, such that the intersection is prevented. For example, a treatment element, like an implant, may automatically be moved from the test position, at which an intersection occurs, to the adjusted position.

For example, the outputting of the adjusted position comprises a highlighting of the adjusted position in the graphical visualization of the three-dimensional digital tissue model with the maxillary and mandibular tissue arranged in the relative positions relative to each other using the graphical user interface.

By highlighting the adjusted position in the graphical visualization, the adjusted position may be indicated as a recommendation for the user. For example, only a position may be highlighted, e.g., by highlighting a position on a surface of the three-dimensional digital tissue model coinciding with the adjusted position. For example, the highlighting of the adjusted position may comprise visualizing the treatment elements, e.g., using three-dimensional digital models of the treatment elements, arranged at the adjusted position.

For example, the determining of the planning adjustment comprises determining an adjusted orientation as a replacement for the test orientation, for which an intersection of the three-dimensional workspace with tissue of the three-dimensional digital tissue model opposite of the test orientation is prevented. The adjusted orientation is output.

Examples may have the beneficial effect that a recommendation for a replacement for the test orientation in form of the adjusted orientation is output. For this adjusted orientation no intersection of the three-dimensional workspace with tissue of the three-dimensional digital tissue model opposite of the adjusted orientation occurs. The determining of the adjusted orientation may, e.g., comprise checking alternative orientations in the vicinity of the test orientation, e.g., within a predefined right circular cone around the test orientation. The test orientation may coincide with an axis of the right circular cone, while the apex of the cone is arranged at a position, for which the test orientation is considered. An angle θ between a generatrix of the cone and the axis of the cone may define a maximum deviation of the alternative orientations from the test orientation. In case of a spatially extended position, for which the test orientation is considered a predefined frustum, e.g., a right conical frustum, may be used to define the alternative orientations being checked. For example, the smaller basis of the predefined frustum may be provided by the spatially position or may comprise the spatially extended position.

As adjusted orientation, e.g., an alternative orientation, at which no intersection occurs and which has a smallest deviation from the test orientation, is being selected. In case no alternative orientation without intersection can be determined within the predefined cone, the angle θ may, e.g., successively be increased, until one or more alternative orientations without intersection are found.

In case the test orientation is the result of a moving of the replacement elements, i.e., is reached by the respective movement, the determining of the adjusted orientation may, e.g., comprise moving further, until an orientation is reach, at which no intersection occurs. The moving of the replacement elements may, e.g., comprise a tilting and/or a rotating around an axis of rotation. Thus, the moving further may comprise a tilting further and/or a rotating further. The moving further may follow a previous direction of movement. For example, the moving further may follow a direction extrapolated from the previous moving, i.e., from a trajectory of the previous moving.

In case the test orientation is the result of a moving of the replacement elements, i.e., is reached by the respective movement, the determining of the adjusted orientation may, e.g., comprise moving back, until an orientation is reach, at which no intersection occurs. The moving of the replacement elements may, e.g., comprise a tilting and/or a rotating around an axis of rotation. Thus, the moving back may comprise a tilting back and/or a rotating back. The moving back may, e.g., follow a trajectory of the previous moving, until an orientation is reach, at which no intersection occurs.

For example, the adjusted orientation may be proposed or automatically executed, i.e., the test orientation may be automatically replaced by the adjusted orientation, such that the intersection is prevented. For example, a treatment element, like an implant, may automatically be moved from the test orientation, for which an intersection occurs, to the adjusted orientation.

For example, the outputting of the adjusted orientation comprises a highlighting of the adjusted orientation in the graphical visualization of the three-dimensional digital tissue model with the maxillary and mandibular tissue arranged in the relative orientations relative to each other using the graphical user interface.

By highlighting the adjusted orientation in the graphical visualization, the adjusted orientation may be indicated as a recommendation for the user. For example, only an orientation may be highlighted, e.g., by highlighting a vector within the three-dimensional digital tissue model coinciding with the adjusted orientation. For example, the highlighting of the adjusted orientation may comprise visualizing the treatment elements, e.g., using three-dimensional digital models of the treatment elements, arranged with the adjusted orientation.

For example, the determining of the planning adjustment comprises determining one or more replacement treatment elements as replacements for one or more of the planned treatment elements. The replacement treatment elements having sizes resulting in an adjusted three-dimensional workspace, for which at the test position with the test orientation an intersection with tissue of the three-dimensional digital tissue model opposite of the test orientation is prevented. An identification signal identifying the one or more replacement treatment elements is output.

Examples may have the beneficial effect that alternative treatment elements as replacements for the selected treatment elements may be suggested. Using these replacement treatment elements may have the beneficial effect that an intersection may be avoided. The intersection may, e.g., be avoided without changing the test position and/or test orientation. For example, the replacement treatment elements may be used in combination with a changing of the test position and/or test orientation.

Alternative treatment elements to be used for executing the intraoral treatment may be proposed or automatically used for the further planning. These alternative treatment elements may have forms, sizes and/or lengths different from the forms, sizes and/or lengths of the selected treatment elements, such that the intersection is prevented. These alternative treatment elements may result in a change of the form, size and/or length of the three-dimensional working space, such that the intersection is prevented.

For example, bent or angulated, smaller and/or shorter alternative treatment elements may be used as replacement treatment elements. For example, a different type of treatment element may be suggested as replacement treatment elements.

For example, the outputting of the one or more replacement treatment elements comprises outputting a graphical visualization of the one or more replacement treatment elements within the three-dimensional digital tissue model with the maxillary and mandibular tissue arranged in the relative positions relative to each other using the graphical user interface. The one or more replacement treatment elements are arranged at the test position and aligned with the test orientation.

For example, the replacement treatment elements may be visualized using three-dimensional digital models of the replacement treatment elements. These three-dimensional digital models of the replacement treatment elements may be arranged at the test position and aligned with the test orientation relative to the three-dimensional digital tissue model.

For example, the one or more selected treatment elements comprise one or more treatment tools.

For example, the one or more treatment tools comprise one or more of the following: an implant drill, an implant drill handle, a drill guide handle, a sleeve, a surgery guide, an implant contra-angle.

An implant drill refers to a drill, i.e., drill head configured for drilling a hole into an oral tissue, e.g., bone tissue for inserting an implant. Implant drills may have different forms configured for different purposes. For drilling an implant hole, different types of implant drills may be used, e.g., a pilot drill, a threaded drill, a tapered drill, and/or a threaded tapered drill. A pilot drill is configured for drilling a pilot hole. A threaded drill is configured for generating a hole with a threaded inner surface. A tapered drill is configured to generate a tapered hole. A threaded tapered drill is configured to generate a tapered hole with a threaded inner surface.

An implant drill handle refers to a handle configured for handling an implant drill. A dental drill handle or dental drill handpiece is a hand-held, mechanical instrument used to perform a variety of dental procedures. An implant drill handle or implant drill handpiece is a dental drill handle or dental drill handpiece configured for implant related procedures. The handpiece itself, e.g., consists of internal mechanical components which initiate a rotational force and provide power to an instrument, e.g., an implant drill. For example, a light source and/or a cooling water-spray system may be incorporated into the handpiece.

A drill guide handle is a handle configured for guiding an implant drill. The drill guide handle may, e.g., comprise a through-hole with a predefined diameter configured for guiding a drill guide of a certain diameter. A sleeve may be configured for guiding an implant drill. Such a sleeve may, e.g., be arranged in a surgery guide. A surgery guide refers to a guide to be arranged on intraoral tissue and configured to provide guidance for a surgery tool. A surgery guide may, e.g., be configured as a drilling guide. A drilling guide defines positions of holes to be drilled into the jawbone, e.g., for inserting implants for fastening a restoration, like a dental bridge or a crown, to the patient's jaw. In addition to defining the position of the holes, the drilling guide may, e.g., provide a guidance for a drilling tool. This guidance may, e.g., be provided by a sleeve implemented in the drilling guide. It may, e.g., also define a depth up to which the drilling tool is to be inserted into the drilling guide, thereby restricting the depths of the holes being drilled.

An implant contra-angle refers to an angled instrument that is configured for implant related procedures, e.g., inserting implants into a hole drilled into a jawbone. The instrument compromises an angle between a base section and a head section. Depending on the operation, different, e.g., rotating instruments be clamped into a reception comprised by the head section. The implant contra-angle may be configured as an implant drill handle. The implant contra-angle may be configured as a torque controlled dental wrench to be used for screwing an implant into place at a precise torque so as not to overload the surrounding bone. For ensuring the precise torque, e.g., a surgical motor may be used, which provides an electronic controlled torque-limitation.

For example, the one or more selected treatment elements comprise one or more dental elements.

For example, the one or more dental elements comprise one or more of the following: an implant, an abutment, a screw, a crown, a bridge.

An implant refers to a prosthesis that interfaces with the bone of a jaw to support a dental prosthesis such as a crown, bridge, denture, or facial prosthesis or to act as an orthodontic anchor. An abutment is a connecting element configured for connecting a dental prosthesis to an implant. A screw refers to a fastening means configured for fastening an abutment and/or a dental prosthesis to an implant.

A crown refers to dental prosthesis, which mimics a natural tooth crown and may be used to replace a natural tooth. It may be connected to implants using abutments, also referred to as connectors, and a fastening means, like screws. Alternatively, the crown may, e.g., be bonded to the abutments using bonding material.

A bridge refers to dental prosthesis in form of a permanent appliance used to replace one or more missing teeth. A dental bridge comprises a plurality of artificial dental elements that are fused together, e.g., one or more artificial teeth are definitively joined to adjacent teeth. It may be connected to implants using abutments, also referred to as connectors, and a fastening means, like screws. Alternatively, the bridge may, e.g., be bonded to the abutments using bonding material.

For example, the method further comprises providing data for controlling a manufacturing of one or more treatment elements to be manufactured. The data defines one or more three-dimensional digital models of one or more treatment elements to be manufactured as templates for the one or more treatment elements to be manufactured. For example, the method further comprises manufacturing of the one or more treatment elements using the data provided for controlling the manufacturing with the manufactured treatment elements being physical copies of the templates defined by the provided data. The one or more treatment elements to be manufactured, e.g., comprise one or more of the selected treatment elements. The one or more treatment elements to be manufactured, e.g., comprise one or more of the replacement treatment elements.

For example, the method further comprises providing data for controlling a manufacturing of one or more of the selected treatment elements. The data defines one or more three-dimensional digital models of the one or more selected treatment elements to be manufactured as templates for the one or more of the selected treatment elements to be manufactured. For example, the method further comprises manufacturing of the one or more of the selected treatment elements using the data provided for controlling the manufacturing with the manufactured treatment elements being physical copies of the templates defined by the provided data.

For example, the method further comprises providing data for controlling a manufacturing of one or more of the replacement treatment elements identified by the identification signal. The data defines one or more three-dimensional digital models of the one or more replacement treatment elements to be manufactured as templates for the one or more replacement treatment elements to be manufactured. For example, the method further comprises manufacturing of the one or more replacement treatment elements using the data provided for controlling the manufacturing with the manufactured replacement treatment elements being physical copies of the templates defined by the provided data.

For the manufacturing of the treatment elements, e.g., computer-controlled additive and/or subtractive methods may be used. For example, the treatment elements may be manufactured using one of the following: machining, 3D printing, casting.

Examples may have the beneficial effect, that treatment elements may be manufactured using a machining device configured to manufacture the treatment elements by processing a blank. For example, the physical preparation guide may be manufactured using a 3D printing device, i.e., a printer, configured to print the respective physical preparation guide. For example, a computer-controlled additive and/or subtractive method may be used for manufacturing a casting matrix configured for casting the physical preparation guide by inserting casting material into the casting matrix and curing the inserted casting material.

For example, the one or more manufactured treatment elements comprise one or more treatment tools, e.g., one or more implant drills, one or more sleeves, one or more surgery guides. For example, the one or more manufactured treatment elements comprise one or more dental elements, e.g., one or more implants, one or more abutments, one or more screws, one or more crowns, one or more bridges.

In another aspect, the invention relates to a computer program product for planning of an intraoral treatment for a patient. The computer program product comprises a non-transitory computer readable storage medium having program instructions embodied therewith. The program instructions are executable by a processor of a computer device to cause the computer device to receive a three-dimensional digital tissue model of intraoral tissue of the patient's oral cavity comprising maxillary and mandibular tissue. Further, a maximum mouth opening value descriptive of a maximum degree of mouth opening achievable by the patient is received. Relative positions of the maxillary and mandibular tissue of the three-dimensional digital tissue model relative to each other are determined for a maximum mouth opening of the patient using the maximum mouth opening value. Size information descriptive of sizes of one or more treatment elements selected to be used for executing the intraoral treatment are received. A three-dimensional working space required for arranging and using the one or more selected treatment elements for executing the intraoral treatment is determined using the size information of the one or more selected treatment elements.

For a test position and test orientation of the one or more selected treatment elements within the three-dimensional digital tissue model with the maxillary and mandibular tissue arranged at the determined relative positions it is checked, whether the working space arranged at the test position and aligned with the test orientation intersects with tissue of the three-dimensional digital tissue model opposite of the test position. In response to a detection of an intersection, an indication signal indicating the detected intersection is output and a planning adjustment for the planning of the intraoral treatment configured to prevent the detected intersection is determined.

The program instructions provided by the computer program product may, e.g., be executable by the processor of the computer device to cause the computer device to execute any of the aforementioned examples of the method for planning an intraoral treatment for a patient.

In another aspect, the invention relates to a computer program for planning of an intraoral treatment for a patient. The computer program comprises program instructions executable by a processor of a computer device to cause the computer device to receive a three-dimensional digital tissue model of intraoral tissue of the patient's oral cavity comprising maxillary and mandibular tissue. Further, a maximum mouth opening value descriptive of a maximum degree of mouth opening achievable by the patient is received. Relative positions of the maxillary and mandibular tissue of the three-dimensional digital tissue model relative to each other are determined for a maximum mouth opening of the patient using the maximum mouth opening value. Size information descriptive of sizes of one or more treatment elements selected to be used for executing the intraoral treatment are received. A three-dimensional working space required for arranging and using the one or more selected treatment elements for executing the intraoral treatment is determined using the size information of the one or more selected treatment elements.

For a test position and test orientation of the one or more selected treatment elements within the three-dimensional digital tissue model with the maxillary and mandibular tissue arranged at the determined relative positions it is checked, whether the working space arranged at the test position and aligned with the test orientation intersects with tissue of the three-dimensional digital tissue model opposite of the test position. In response to a detection of an intersection, an indication signal indicating the detected intersection is output and a planning adjustment for the planning of the intraoral treatment configured to prevent the detected intersection is determined.

The program instructions provided by the computer program may, e.g., be executable by the processor of the computer device to cause the computer device to execute any of the aforementioned examples of the method for planning an intraoral treatment for a patient.

In another aspect, the invention relates to a computer device for planning of an intraoral treatment for a patient. The computer device comprises a processor and a memory storing program instructions executable by the processor. Execution of the program instructions by the processor causes the computer device to receive a three-dimensional digital tissue model of intraoral tissue of the patient's oral cavity comprising maxillary and mandibular tissue. Further, a maximum mouth opening value descriptive of a maximum degree of mouth opening achievable by the patient is received. Relative positions of the maxillary and mandibular tissue of the three-dimensional digital tissue model relative to each other are determined for a maximum mouth opening of the patient using the maximum mouth opening value. Size information descriptive of sizes of one or more treatment elements selected to be used for executing the intraoral treatment are received. A three-dimensional working space required for arranging and using the one or more selected treatment elements for executing the intraoral treatment is determined using the size information of the one or more selected treatment elements.

For a test position and test orientation of the one or more selected treatment elements within the three-dimensional digital tissue model with the maxillary and mandibular tissue arranged at the determined relative positions it is checked, whether the working space arranged at the test position and aligned with the test orientation intersects with tissue of the three-dimensional digital tissue model opposite of the test position. In response to a detection of an intersection, an indication signal indicating the detected intersection is output and a planning adjustment for the planning of the intraoral treatment configured to prevent the detected intersection is determined.

Execution of the program instructions by the processor of the computer device may, e.g., cause the computer device to execute any of the aforementioned examples of the method for planning an intraoral treatment for a patient.

For example, the computer device is comprised by a manufacturing system. The computer device may be any of the aforementioned examples of a computer device. The manufacturing system may further comprise one or more manufacturing devices configured to manufacture one or more treatment elements to be manufactured. The treatment elements to be manufactured may, e.g., comprise one or more of the selected treatment elements. The manufactured treatment elements to be manufactured may, e.g., comprise one or more of the replacement treatment elements.

Execution of the program instructions by the processor further causes the computer device to control the one or more manufacturing devices to manufacture the one or more treatment elements to be manufactured using the data provided for controlling the manufacturing with the manufactured treatment elements being physical copies of the templates defined by the provided data. The one or more treatment elements being manufactured, e.g., comprise one or more of the selected treatment elements. The one or more treatment elements being manufactured, e.g., comprise one or more of the replacement treatment elements.

For example, the one or more manufactured treatment elements comprise one or more treatment tools, e.g., one or more implant drills, one or more sleeves, one or more surgery guides. For example, the one or more manufactured treatment elements comprise one or more dental elements, e.g., one or more implants, one or more abutments, one or more screws, one or more crowns, one or more bridges.

For the manufacturing of the treatment elements, e.g., computer-controlled additive and/or subtractive methods may be used. For example, the treatment elements may be manufactured using one of the following: machining, 3D printing, casting.

For example, the one or more manufacturing devices of the manufacturing system may comprise one or more of the following: a machining device, a 3D printing device.

Examples may have the beneficial effect, that treatment elements may be manufactured using a machining device configured to manufacture the treatment elements by processing a blank. For example, the physical preparation guide may be manufactured using a 3D printing device, i.e., a printer, configured to print the respective physical preparation guide. For example, a computer-controlled additive and/or subtractive method may be used for manufacturing a casting matrix configured for casting the physical preparation guide by inserting casting material into the casting matrix and curing the inserted casting material.

It is understood that one or more of the above-described examples and embodiments may be combined as long as the combined embodiments are not mutually exclusive.

### BRIEF DESCRIPTION OF THE DRAWINGS

In the following, examples are described in greater detail making reference to the drawings in which:

FIG. 1 shows an exemplary measuring of a maximum mouth opening value;

FIG. 2 shows exemplary three-dimensional digital models of treatment elements arranged in an exemplary three-dimensional digital tissue model;

FIG. 3 shows exemplary three-dimensional digital models of treatment elements arranged in an exemplary three-dimensional digital tissue model;

FIG. 4 shows an exemplary three-dimensional digital tissue model with an exemplary three-dimensional working space arranged therein;

FIG. 5 shows an exemplary three-dimensional digital tissue model with a further exemplary three-dimensional working space arranged therein;

FIG. 6 shows exemplary alternative positions in the vicinity of a test position and alternative orientations in the vicinity of a test orientation;

FIG. 7 shows an exemplary digital articulator with an exemplary three-dimensional digital tissue model;

FIG. 8 shows exemplary treatment elements;

FIG. 9A shows a first part of a flowchart illustrating an exemplary method for planning an intraoral treatment;

FIG. 9B shows a second part of a flowchart illustrating an exemplary method for planning an intraoral treatment;

FIG. 10A shows a first part of another flowchart illustrating an exemplary method for planning an intraoral treatment;

FIG. 10B shows a second part of another flowchart illustrating an exemplary method for planning an intraoral treatment;

FIG. 11 shows a flowchart illustrating an exemplary method for manufacturing a treatment element;

FIG. 12 shows a flowchart illustrating a further exemplary method for manufacturing a treatment element;

FIG. 13 shows an exemplary computer device for planning an intraoral treatment;

FIG. 14 shows an exemplary computer device for planning an intraoral treatment; and

FIG. 15 shows an exemplary system for manufacturing a treatment element.

### DETAILED DESCRIPTION

In the following, similar elements are denoted by the same reference numerals. Elements which have been discussed previously will not necessarily be discussed in later figures if the function is equivalent.

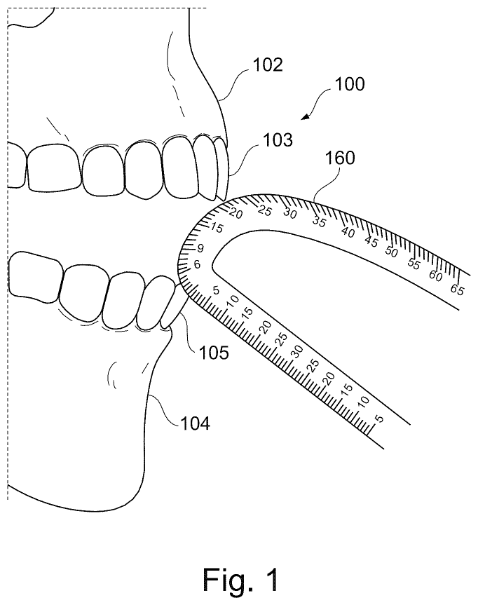

FIG. 1 shows an exemplary measuring of a maximum mouth opening value. The maximum mouth opening value is descriptive of a maximum degree of mouth opening achievable by a patient. In case of FIG. 1, e.g., a maximal interincisal opening (MIO) measurement is executed for determining the maximum mouth opening value. MIO measures the distance between the central incisors 103, 105, when a patient's mouth is fully open. To determine a patient's MIO, e.g., a special scale 160 may be used indicating a quantitative range of how wide a patient can open the mouth. The patient opens the mouth as wide as possible and the distance between mandible 104 and maxilla 102 is measured in form of a distance between the central mandibular incisors 105 and the central maxillary incisors 103.

Alternatively, e.g., a range of motion (ROM) measurement may be used, which determines a maximum distance the mandible moves. The results of MIO and ROM may be the same, but they can also be different, e.g., in case of a patient with an open bite.

Alternatively, e.g., the maximum mouth opening value may, e.g., be determined as part of jaw motion tracking data tracking jaw movements of the patient. For acquiring the jaw motion tracking data, e.g., an electronic jaw movement registration and tracking system may be used. The jaw movement registration and tracking system may, e.g., comprise an electronic facebow, which is used to register and track movements of the patient's jaws. The jaw movement registration and tracking system may, e.g., be configured for register and track movements of the patient's jaws using a motion capture method. The motion capture method may, e.g., use marker, like magnets, for capturing the motion. The motion capture method may, e.g., use a markerless approach, e.g., with one or more optical sensors configured for optically capturing jaw movements.

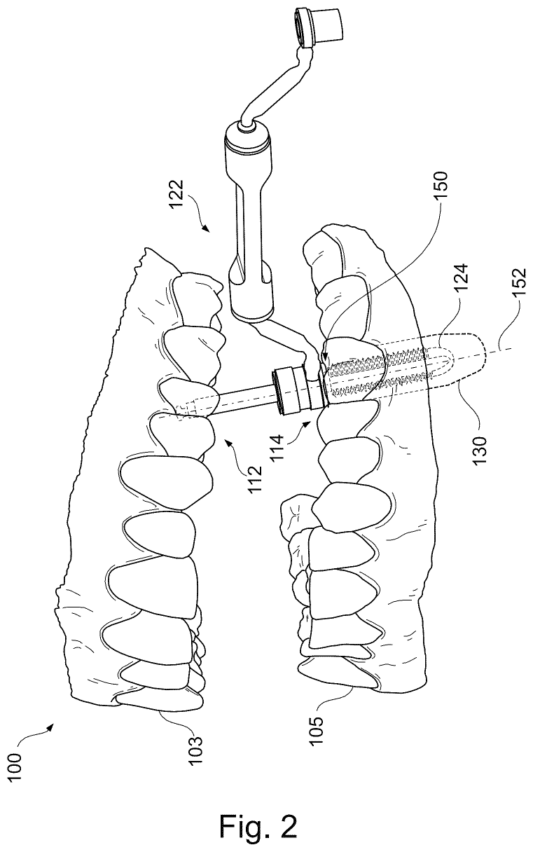

FIG. 2 shows an exemplary three-dimensional digital tissue model 100 of intraoral tissue of the patient's oral cavity comprising maxillary tissue 112 and mandibular tissue 114. In FIG. 2, the three-dimensional digital tissue model 100 comprises a three-dimensional digital model of a maxilla, i.e., maxillary tissue 112, and a three-dimensional digital model of a mandible, i.e., the mandible tissue 114. Maxilla tissue 112 and mandible tissue 114 are arranged relative to each other at relative positions such that a distance between the maxilla tissue 112 and the mandible tissue 114 corresponds to a maximum mouth opening value measured for the patient. The maximum mouth opening value may, e.g., be a MIO value measured as illustrated in FIG. 1. In this case, the maxilla tissue 112 and mandible tissue 114 are arranged at relative positions such that a distance between the central incisors 103, 105 of maxilla and mandible corresponds to the MIO value measured. For arranging the maxilla tissue 112 and the mandible tissue 114 at the relative positions, e.g., a digital articulator and/or jaw motion tracking data may be used.

Furthermore, three-dimensional digital models 122 of selected treatment elements selected to be used for executing the intraoral treatment are arranged in the three-dimensional digital tissue model 100. In case of FIG. 2, the selected treatment elements of the three-dimensional digital models 122 may, e.g., be treatment elements for inserting an implant 124 into a hole 130 drilled into the mandible tissue 114 at a treatment side. The treatment elements of the three-dimensional digital models 122 are arranged at the treatment side at a test position 150 with a test orientation 152, i.e., at a position with an orientation to be checked.

In case of FIG. 2, the patient may have open mouth limitation resulting in a limited achievable maximum mouth opening. In case of FIG. 2, the selected treatment elements of the three-dimensional digital models 122 intersect with the maxilla tissue 112, when being arranged at the test position 150 with the test orientation 152. Thus, the selected treatment elements may not be usable at the test position 150 with the test orientation 152.

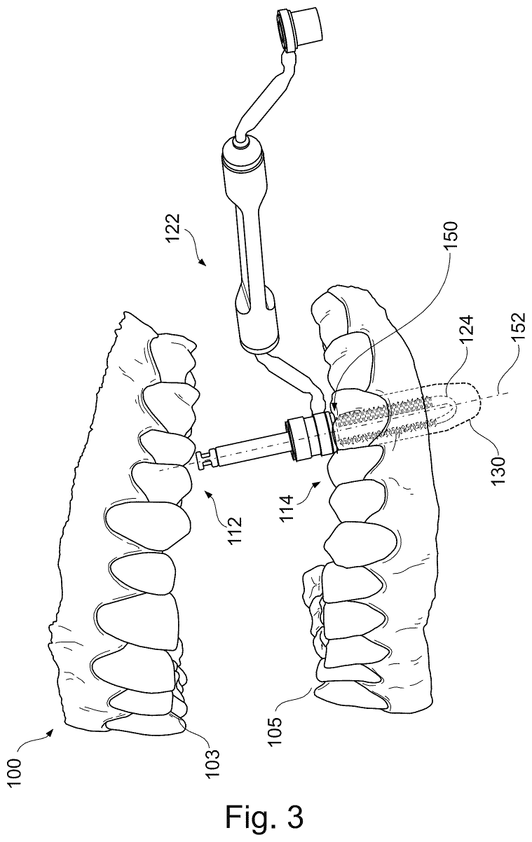

FIG. 3 shows the exemplary three-dimensional digital tissue model 100 of FIG. 2 with the exemplary three-dimensional digital models 122 of treatment elements of FIG. 2. In case of FIG. 3 the maximum mouth opening value, e.g., a MIO value, is larger than the maximum mouth opening value of FIG. 2. Thus, the maxilla tissue 112 and mandible tissue 114 in FIG. 3 are further spaced apart from each other than in FIG. 2. In case of FIG. 3, due to the larger maximum mouth opening value, the three-dimensional digital models 122 of treatment elements can be arranged at the same test position 150 with the same test orientation 152 as in FIG. 2 without intersecting with the maxilla tissue 112. Thus, for the maximum mouth opening value of FIG. 3, the selected treatment elements 122 are usable at the test position 150 with the test orientation 152.

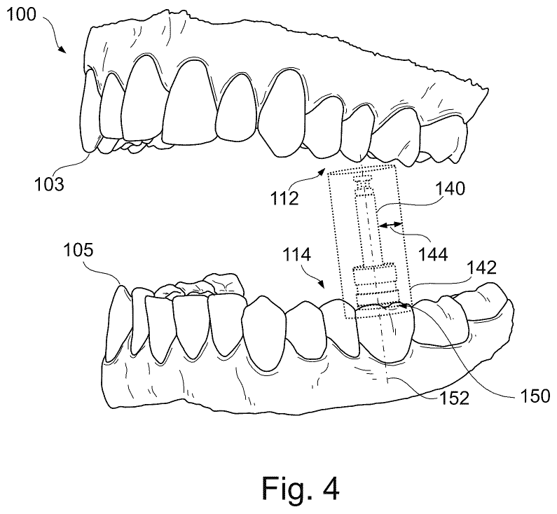

FIG. 4 shows an exemplary three-dimensional digital tissue model 100 of intraoral tissue of the patient's oral cavity comprising maxillary tissue 112 and mandibular tissue 114. In FIG. 4, the three-dimensional digital tissue model 100 comprises a three-dimensional digital model of a maxilla tissue 112 and a three-dimensional digital model of a mandible tissue 114. Maxilla tissue 112 and mandible tissue 114 are arranged relative to each other at relative positions such that a distance between maxilla tissue 112 and mandible tissue 114 corresponds to a maximum mouth opening value measured for the patient. The maximum mouth opening value may, e.g., be a MIO value measured as illustrated in FIG. 1. In this case, the maxilla tissue 112 and mandible tissue 114 are arranged at relative positions such that a distance between the central incisors 103, 105 of maxilla tissue 112 and mandible tissue 114 corresponds to the MIO value measured. For arranging the maxilla tissue 112 and mandible tissue 114 at the relative positions, e.g., a digital articulator may be used and/or jaw motion tracking data may be used.

Furthermore, a three-dimensional working space 140 required for arranging and using one or more selected treatment elements for executing an intraoral treatment is shown. The three-dimensional working space 140 is determined using size information descriptive of sizes of the one or more treatment elements selected to be used for executing the intraoral treatment. For example, the three-dimensional working space 140 may correspond to the form of the selected treatment elements to be arranged between the maxilla tissue 112 and mandible tissue 114 for the planned treatment. Additionally, a clearance 144 required for a moving and positioning of the one or more selected treatment elements at the test position 150 with the test orientation 152 may be taken into account. The additional clearance 144 may result in an extended three-dimensional working space 142. The three-dimensional working space 140 may, e.g., be extended in different directions and/or at different sections by different amount resulting in an extended three-dimensional working space 142 of a basic geometric form, like a cylinder, e.g., a circular right cylinder as shown in FIG. 3. The basic geometric form could also have the form of a circular oblique cylinder depending on the test orientation 152. Alternatively, the three-dimensional working space 140 may, e.g., be extended in all directions and/or at all sections by the same amount, i.e., scaled up resulting in an extended three-dimensional working space 142 having the same form as the three-dimensional working space 140, but a larger size (not shown).

The three-dimensional working space 140, 142 may be used for testing potential positions 150 and/or orientations 152 for the one or more selected treatment elements. For example, the one or more selected treatment elements may comprise an implant. Thus, potentials positions and/or orientations of the implant may be checked, when planning an implant. The three-dimensional working space 140, 142 may be a space arranged on the intraoral tissue 100 at a position 150 and with an orientation 152 required for executing the treatment at the respective position 150. In case of an implant, the three-dimensional working space 140, 142 may, e.g., be position on the intraoral tissue 100 at a position 150, where a hole for the implant may be drilled, and with an orientation 152, e.g., aligned parallel with a length axis of the implant.

Depending on the position 150 and orientation 152 as well as the form and size of the three-dimensional working space 140, 142, the three-dimensional working space 140, 142 may intersect with tissue of the three-dimensional digital tissue model 100 opposite of the test position 150, in case of FIG. 4 with the maxilla tissue 112. In FIG. 4, there is no intersection of the three-dimensional working space 140, 142 with the maxilla tissue 112.

For the test position 150 with the test orientation 152 of the three-dimensional working space 140, 142 it may be checked whether the working space 140, 142 arranged at the test position 150 with the test orientations 152 intersects with tissue of the three-dimensional digital tissue model 100 opposite of the test position 150, i.e., the maxilla tissue 112. In response to a detection of an intersection, an indication signal indicating the detected intersection may be output and a planning adjustment for the planning of the intraoral treatment determined, which is configured to prevent the detected intersection. For example, a section of the three-dimensional working space 140, 142 intersecting with the maxilla tissue 112 and/or a section of the maxilla tissue 112 being intersected by the three-dimensional working space 140, 142 may be highlighted.

For example, the determined planning adjustment may compromise a restriction of a selectability of the positions 150 and orientations 152 for the treatment elements, i.e., the three-dimensional digital models 122, and thus for the working space 140, 142. The test position 150 and/or test orientation 152 may be excluded from the selectability for the planned treatment, i.e., there combination of position 150 and orientation 152 of the working space 140, 142, for which the intersection is detected, may not be selectable, when further planning the treatment.

For example, a graphical visualization of the treatment elements, i.e., the three-dimensional digital models 122 of the selected treatment elements, is output using a graphical user interface. The determined planning adjustment may comprise a restriction of a movability of the three-dimensional digital models 122 relative to the three-dimensional digital tissue model 100. For example, a moving of the treatment elements to the test position 150 and into alignment with the test orientation 152, for which the intersecting was detected, may be blocked.

For example, the determining of the planning adjustment comprises determining an adjusted position and/or orientation as a replacement for the test position 150 and with the test orientation 152, at which an intersection of the three-dimensional workspace 140, 142 with tissue of the three-dimensional digital tissue model 100 opposite of the adjusted position 150 is prevented, e.g., with the maxilla tissue 112. The adjusted position and/or orientation may be output. For example, the outputting of the adjusted position comprises and/or a highlighting of the adjusted position and/or orientation in the graphical visualization of the three-dimensional digital tissue model 100 using the graphical user interface. Thus, the output of the adjusted position and/or orientation may be provided as an automatic alternative suggestion for preventing an intersecting.

For example, the determining of the planning adjustment comprises determining one or more replacement treatment elements as replacements for one or more of the selected treatment elements represented by the three-dimensional digital models 122 and used for the planning. The replacement treatment elements may have sizes resulting in an adjustment of three-dimensional workspace 140, 142, e.g., a reduction in size and/or adjusted form, for which at the test position 150 with the test orientation 152 an intersection with the maxilla 112 is prevented. An identification signal identifying the one or more replacement treatment elements may be output. For example, the outputting of the one or more replacement treatment elements comprises outputting a graphical visualization of the one or more replacement treatment elements within the three-dimensional digital tissue model 100. For example, three-dimensional digital models 122 of the one or more replacement treatment elements may be output. The one or more replacement treatment elements may be arranged at the test position 150 and aligned with the test orientation 152.

For example, a set of suggested replacement treatment elements and/or of suggested combinations of replacement treatment elements is provided, e.g., displayed on a graphical user interface. The use may select on or more of the suggested replacement treatment elements or a suggested combination of replacement treatment elements form the set. The one or more selected replacement treatment elements or the selected combination of replacement treatment elements may then be arranged at the test position 150 and aligned with the test orientation 152.

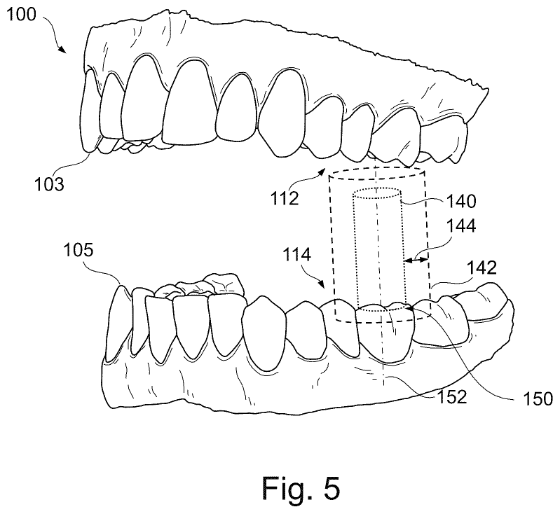

FIG. 5 shows an exemplary three-dimensional digital tissue model 100 of intraoral tissue of the patient's oral cavity comprising maxillary tissue 112 and mandibular tissue 114. The three-dimensional digital tissue model 100 of FIG. 5 corresponds to the three-dimensional digital tissue model 100 of FIG. 4. In addition, a three-dimensional working space 140 required for arranging and using one or more selected treatment elements for executing an intraoral treatment is shown. The three-dimensional working space 140 is determined using size information descriptive of sizes of the one or more treatment elements selected to be used for executing the intraoral treatment. Further, the three-dimensional working space 140 is arranged at a test position 150 with a test orientation 152. The three-dimensional working space 140 shown in FIG. has a basic geometric form, like a cylinder, e.g., a circular right cylinder. The basic geometric form could also have the form of a circular oblique cylinder depending on the test orientation 152. The size of the basic geometric form may, e.g., be adjusted such that it encloses the selected treatment elements to be arranged between the maxilla tissue 112 and mandible tissue 114 for the planned treatment. Additionally, a clearance 144 required for a moving and positioning of the one or more selected treatment elements at the test position 150 with the test orientation 152 may be taken into account. The three-dimensional working space 140 may, e.g., be scaled up resulting in an extended three-dimensional working space 142 as shown in FIG. 5 having the same form as the three-dimensional working space 140, but a larger.

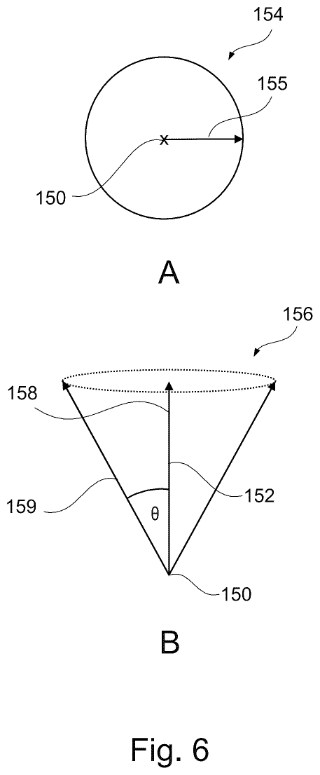

FIG. 6 illustrates exemplary alternative positions in the vicinity of a test position 150 in upper drawing A, and alternative orientations in the vicinity of a test orientation 152 in lower drawing B. For example, an intersection may be detected for a test position 150. For example, for determining a replacement position for a test position 150 in form of the adjusted position, at which no intersection of the three-dimensional workspace with tissue of the three-dimensional digital tissue model opposite of the adjusted position occurs, alternative positions in the vicinity of the test position 150 may be checked. For example, alternative positions within a region 154 defined by a predefined radius 155 around the test position 150 may be checked. As adjusted position, e.g., a nearest alternative position is being selected, at which no intersection occurs. In case no alternative position without intersection can be determined within the predefined radius 155, the radius 155 may, e.g., successively be extended, until one or more alternative positions without intersection are found.

For example, an intersection may be detected for a test orientation 152. For example, for determining a replacement orientation for a test orientation 152 in form of the adjusted orientation, for which no intersection of the three-dimensional workspace with tissue of the three-dimensional digital tissue model opposite of the adjusted position occurs, alternative orientations in the vicinity of the test orientation 152 may be checked. For example, alternative orientations within a predefined right circular cone 156 around the test orientation 152 may be checked. The test orientation 152 may coincide with an axis 158 of the right circular cone 156, while the apex of the cone 156 is arranged at a position, e.g., a test position 150, for which the test orientation 152 is considered. An angle θ between a generatrix 159 of the cone 156 and the axis 158 of the cone 156 may define a maximum deviation of the alternative orientations from the test orientation 152.

As adjusted orientation, e.g., an alternative orientation, at which no intersection occurs and which has a smallest deviation from the test orientation 152, is being selected. In case no alternative orientation without intersection can be determined within the predefined cone 156, the angle θ may, e.g., successively be increased, until one or more alternative orientations without intersection are found.

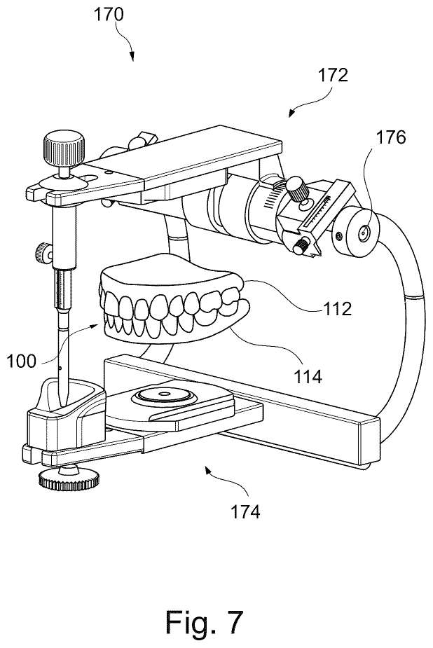

FIG. 7 illustrates a digital articulator 170, which may be used for determining the relative positions of the maxillary and mandibular tissue 112, 114 of the three-dimensional digital tissue model 100 relative to each other. The digital articulator 170 is configured to reproduce some or all the movements of the mandibular tissue 114 in relation to the maxillary tissue 114. The digital articulator 170 may be configured to simulate the position and movements of the bilateral temporomandibular joints, which determine the relative movements of mandible and maxilla. The digital articulator 170 may, e.g., be configured to mimic individual movements of the maxillary and mandibular tissue 112, 114 relative to each other defined by tracking information, which is acquired by tracking movements of a patient's jaws. The digital articulator 170 may, e.g., be configured to mimic movements of the maxillary and mandibular tissue 112, 114 defined by averaged moving information of human jaw movements.

The digital articulator 170 may, e.g., provided in form of a three-dimensional digital model of an articulator comprising an upper articulator part 172 and a lower articulator part 174 connected with each other via a hinge 176. A position of the maxillary tissue 112 relative to the upper articulator part 172 and a position of the mandibular tissue 114 relative to the lower articulator part 174 may be fixed, such that a movement of the upper articulator part 172 relative to the lower articulator part 174 using the hinge 176 may result in a relative movement of the maxillary and mandibular tissue 112, 114 mimicking a movement of the patient's mandible and maxilla.

The relative position between the maxillary and mandibular tissue 112, 114 within the digital articulator 170 may, e.g., be determined using a measurement of a relative position of the patient's maxillary and mandibular tissue in the oral cavity. For this measurement, e.g., a facebow may be used. The digital articulator 170 may then, e.g., be opened with the maxillary and mandibular tissue 112, 114 arranged therein simulating an opening of the patient's mouth. The digital articulator may, e.g., be opened until the maximum degree of mouth opening described maximum mouth opening value is reached. Thus, anatomically correct relative positions of the maxillary and mandibular tissue 112, 114 of the three-dimensional digital tissue model 100 relative to each other may be determined for the maximum mouth opening of the patient according to the maximum mouth opening value.

For the determining of the relative positions of the maxillary and mandibular tissue 112, 114 relative to each other using the digital articulator, the method may, e.g., further comprise receiving tracking information of a tracking of jaw movements of the patient's and using the received tracking information for defining a movement of the maxillary and mandibular tissue relative to each other with the digital articulator.

For the determining of the relative positions of the maxillary and mandibular tissue relative to each other using the digital articulator, the method may, e.g., further comprises receiving averaged moving information of human jaw movements and using the received moving information for defining the movement of the maxillary and mandibular tissue relative to each other with the digital articulator.

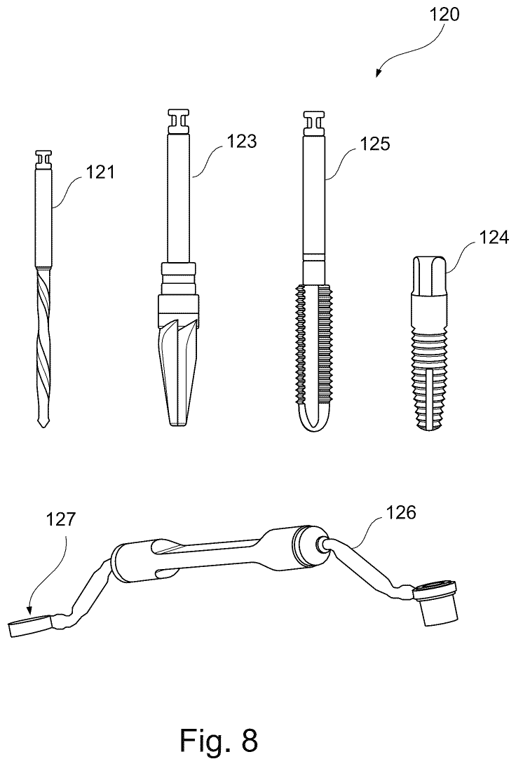

FIG. 8 shows exemplary treatment elements 120. Such exemplar treatment elements 120 may, e.g., comprise treatment tools used for inserting an implant 124. The exemplar treatment elements 120 may, e.g., comprise one or more of the following: a pilot drill 121 configured for drilling an initial hole for the implant 124, a tapered drill 123 for drilling the full hole configured to receive the implant, a threated tapered drill 125 for inserting a threat into the hole matching a thread of the implant 124, the implant 124.

Furthermore, an exemplary treatment element 120 in form of a drill guide handle 126 is shown. Such a drill guide handle 126 is a handle configured for guiding an implant drill. The drill guide handle 126 may, e.g., comprise a through-hole 127 with a predefined diameter configured for guiding a drill guide of a certain diameter.

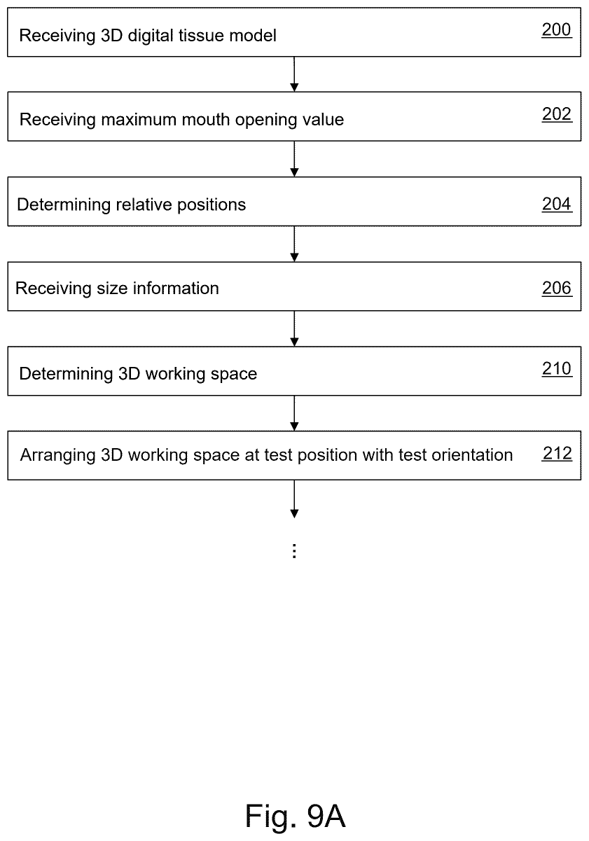

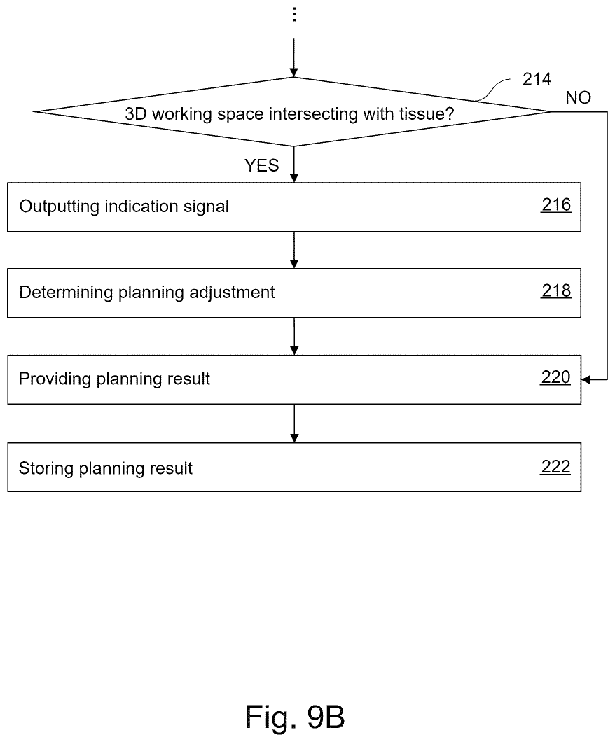

FIG. 9A shows a first part of an exemplary method for planning an intraoral treatment for a patient. In block 200, a three-dimensional digital tissue model of intraoral tissue of the patient's oral cavity comprising maxillary and mandibular tissue is received. For example, the intraoral tissue comprises hard tissue, like, e.g., teeth or bone tissue. For example, the intraoral tissue comprises soft tissue, like, e.g., gingiva tissue. For example, maxillary tissue comprised by the intraoral tissue comprises maxillary hard tissue, like, e.g., maxillary teeth or maxillary bone tissue. For example, mandibular tissue comprised by the intraoral tissue comprises mandibular hard tissue, like, e.g., mandibular teeth or mandibular bone tissue.

In block 202, a maximum mouth opening value descriptive of a maximum degree of mouth opening achievable by the patient is received. For example, the maximum mouth opening value is a value determined using a maximal interincisal opening (MIO) measurement or a range of opening (ROM) measurement. In block 204, relative positions of the maxillary and mandibular tissue of the three-dimensional digital tissue model relative to each other are determined for a maximum mouth opening of the patient using the maximum mouth opening value. For the determining of the relative positions of the maxillary and mandibular tissue relative to each other using the digital articulator, the method, e.g., further comprises receiving tracking information of a tracking of jaw movements of the patient's and using the received tracking information for defining a movement of the maxillary and mandibular tissue relative to each other with the digital articulator. For the determining of the relative positions of the maxillary and mandibular tissue relative to each other using the digital articulator, the method, e.g., further comprises receiving averaged moving information of human jaw movements and using the received moving information for defining the movement of the maxillary and mandibular tissue relative to each other with the digital articulator.

In block 206, size information descriptive of sizes of one or more treatment elements selected to be used for executing the intraoral treatment are received. For example, the size information descriptive of the sizes of the one or more selected treatment elements are received from one or more libraries of treatment elements comprising a plurality of entries assigned to different treatment elements of a plurality of different treatment elements with individual entries of the plurality of entries comprising size information of individual treatment elements of the plurality of treatment elements, to which the individual entries are assigned.

For example, the one or more selected treatment elements comprise one or more treatment tools. For example, the one or more treatment tools comprise one or more of the following: an implant drill, an implant drill handle, a drill guide handle, a sleeve, a surgery guide, an implant contra-angle.

For example, the one or more selected treatment elements comprise one or more dental elements. For example, the one or more dental elements comprise one or more of the following: an implant, an abutment, a screw, a crown, a bridge.

In block 210, a three-dimensional working space required for arranging and using the one or more selected treatment elements for executing the intraoral treatment is determined using the size information of the one or more selected treatment elements. In block 212, the three-dimensional working space is arranged at a test position and aligned with a test orientation. The method is continued in FIG. 9B.

FIG. 9B shows a first part of the exemplary method for planning an intraoral treatment for a patient of FIG. 9A. For the test position and test orientation of the one or more selected treatment elements within the three-dimensional digital tissue model with the maxillary and mandibular tissue arranged at the determined relative positions it is checked in block 214, whether the working space arranged at the test position and aligned with the test orientation intersects with tissue of the three-dimensional digital tissue model opposite of the test position. If the three-dimensional working space intersects with tissue of the three-dimensional digital tissue model opposite of the test position, an indication signal indicating the detected intersection is output in block 216. For example, the outputting of the indication signal comprises a highlighting of the intersection in a graphical visualization of the three-dimensional digital tissue model with the maxillary and mandibular tissue arranged in the relative positions relative to each other. The graphical visualization of the three-dimensional digital tissue model with the highlighted intersection is output using a graphical user interface.

In block 218, a planning adjustment for the planning of the intraoral treatment configured to prevent the detected intersection is determined. The planning adjustment is provided and thus the detected intersection is prevented.

For example, the test position for the one or more selected treatment elements is a position of a plurality of potential position tested for the planned treatment. The determined planning adjustment compromises a restriction of a selectability of the positions of the plurality of positions. The test position is excluded from the selectability for the planned treatment. For example, the test orientation of the one or more selected treatment elements is an orientation of a plurality of potential orientation tested for the planned treatment. The determined planning adjustment compromises a restriction of a selectability of the orientations of the plurality of orientations. The test orientation is excluded from the selectability for the planned treatment.

For example, the method further comprises outputting a graphical visualization of the treatment elements using the graphical user interface. The determined planning adjustment comprises a restriction of a movability of the treatment elements relative to the three-dimensional digital tissue model. One or more of the following is excluded: a moving of the treatment elements to the test position, a moving of the treatment elements into alignment with the test orientation.

For example, the determining of the planning adjustment comprises determining an adjusted position as a replacement for the test position, at which an intersection of the three-dimensional workspace with tissue of the three-dimensional digital tissue model opposite of the adjusted position is prevented. The adjusted position is output. For example, the outputting of the adjusted position comprises a highlighting of the adjusted position in the graphical visualization of the three-dimensional digital tissue model with the maxillary and mandibular tissue arranged in the relative positions relative to each other using the graphical user interface.

For example, the determining of the planning adjustment comprises determining an adjusted orientation as a replacement for the test orientation, for which an intersection of the three-dimensional workspace with tissue of the three-dimensional digital tissue model opposite of the test orientation is prevented. The adjusted orientation is output. For example, the outputting of the adjusted orientation comprises a highlighting of the adjusted orientation in the graphical visualization of the three-dimensional digital tissue model with the maxillary and mandibular tissue arranged in the relative orientations relative to each other using the graphical user interface.

For example, the determining of the planning adjustment comprises determining one or more replacement treatment elements as replacements for one or more of the planned treatment elements. The replacement treatment elements having sizes resulting in an adjusted three-dimensional workspace, for which at the test position with the test orientation an intersection with tissue of the three-dimensional digital tissue model opposite of the test orientation is prevented. An identification signal identifying the one or more replacement treatment elements is output. For example, the outputting of the one or more replacement treatment elements comprises outputting a graphical visualization of the one or more replacement treatment elements within the three-dimensional digital tissue model with the maxillary and mandibular tissue arranged in the relative positions relative to each other using the graphical user interface. The one or more replacement treatment elements are arranged at the test position and aligned with the test orientation.

In block 220, a planning result is provided, which may be adjusted in case of a planning adjustment. If the three-dimensional working space does not intersect with tissue of the three-dimensional digital tissue model opposite of the test position, planning result is provided, which has been successfully cached is provided. The planning result defines, e.g., position and orientation of the one or more selected treatment elements within the three-dimensional digital model, for which the three-dimensional working space does not intersect with tissue of the three-dimensional digital tissue model opposite the position, when the maxillary and mandibular tissue are arranged at the relative positions determined in block 204. In block 222, the provided planning results are stored.

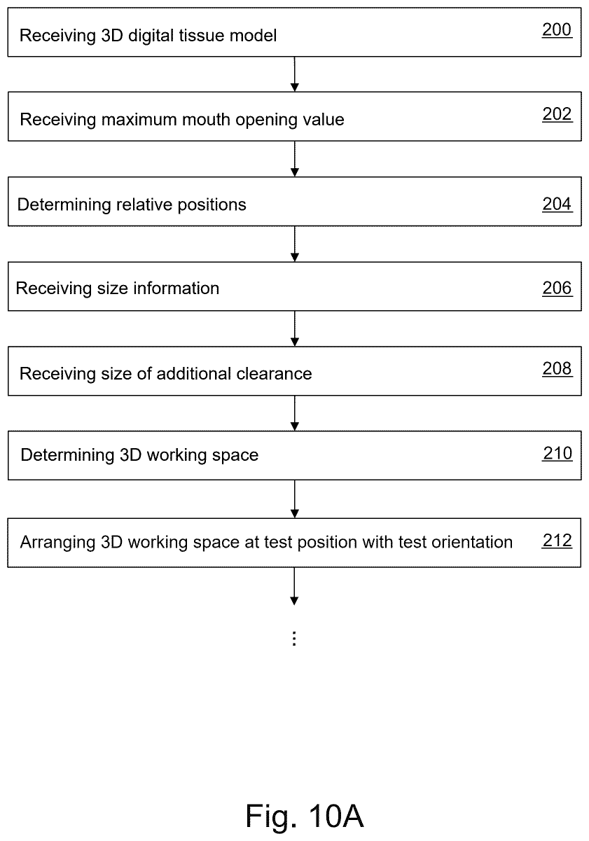

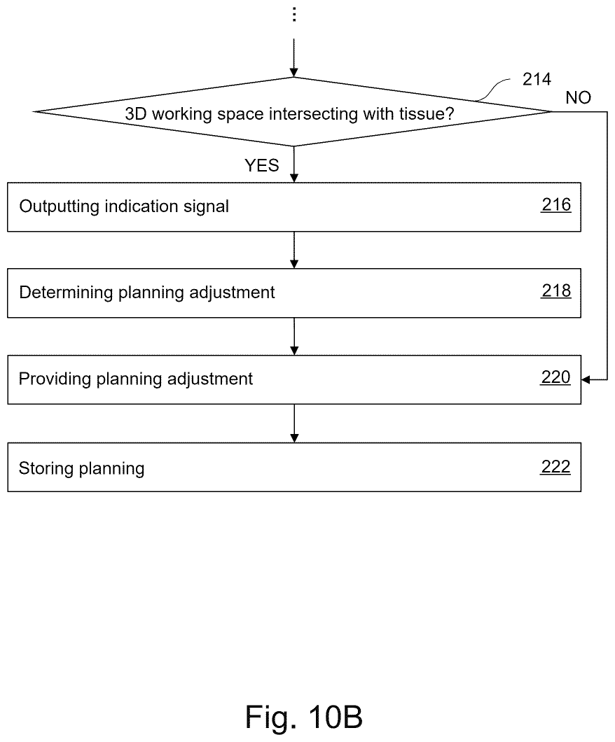

FIG. 10A and FIG. 10B show another exemplary method for planning an intraoral treatment for a patient. FIG. 10A shows a first part of the exemplary method, which is continued in FIG. 8B showing a second part of the exemplary method. Blocks 200 to 222 of FIG. 10A and FIG. 10B correspond to blocks 200 to 222 of FIG. 9A and FIG. 9B. The only difference is that the method of FIG. 10A comprises additional block 208. In block 208, a clearance value defining a size of an additional clearance required for a moving and positioning of the one or more selected treatment elements is received. The clearance value is used for determining the three-dimensional working space in block 210. Thus, additional clearance is taken into account for moving and positioning the one or more selected treatment element at the test position with the test orientation, when the three-dimensional working space is arranged at the test position with the test orientation.

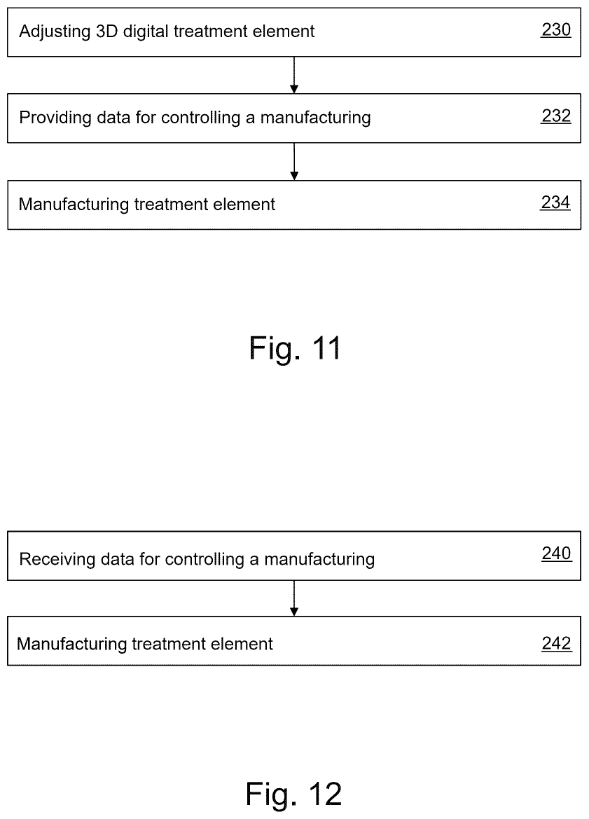

FIG. 11 shows an exemplary method for manufacturing one or more adjusted treatment elements for the intraoral treatment being planned. For example, the one or more selected treatment elements comprise one or more treatment tools. For example, the one or more treatment tools comprise one or more of the following: an implant drill, an implant drill handle, a drill guide handle, a sleeve, a surgery guide, an implant contra-angle. For example, the one or more selected treatment elements comprise one or more dental elements. For example, the one or more dental elements comprise one or more of the following: an implant, an abutment, a screw, a crown, a bridge.

In block 230, the one or more three-dimensional digital treatment elements are adjusted using the adjustment of the planning, e.g., determined in block 218 of FIG. 9B or FIG. 10B. This adjustment comprises an adjusting, e.g. reducing of size, of one or more three-dimensional digital treatment elements. In block 232, data is provided for controlling the manufacturing of the one or more of the treatment elements. The data provided in block 232 comprises the one or more adjusted three-dimensional digital treatment elements resulting from block 230. In block 234, the one or more physical treatment elements are manufactured using the manufacturing data with one or more adjusted more three-dimensional digital treatment elements as templates for the manufacturing. For the manufacturing of the treatment elements, e.g., computer-controlled additive and/or subtractive methods may be used. For example, the treatment elements may be manufactured using one of the following: machining, 3D printing, casting.

FIG. 12 shows an exemplary method for manufacturing one or more selected and/or adjusted treatment elements. In block 240, data for controlling a manufacturing of one or more treatment elements, e.g., comprising one or more of the selected treatment elements and/or one or more replacement treatment elements identified by the identification signal. The received data defines one or more three-dimensional digital models of the treatment elements to be manufactured as templates for the one or more treatment elements to be manufactured. In block 242, the one or more treatment elements are manufactured using the data received in block 240 for controlling the manufacturing with the manufactured treatment elements being physical copies of the templates defined by the received data. For the manufacturing of the treatment elements, e.g., computer-controlled additive and/or subtractive methods may be used. For example, the treatment elements may be manufactured using one of the following: machining, 3D printing, casting.

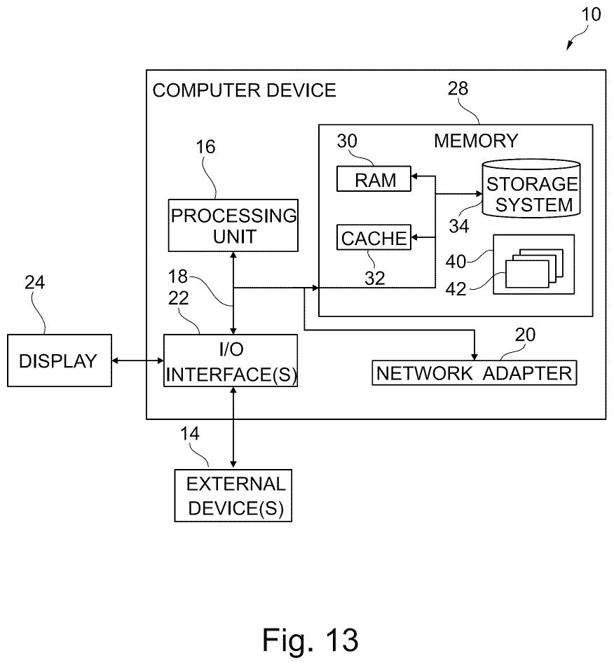

FIG. 13 shows a schematic diagram of an exemplary computer device 10 for planning of an intraoral treatment for a patient. The computer device 10 may be operational with numerous other general-purpose or special-purpose computing system environments or configurations. Computer device 10 may be described in the general context of computer device executable instructions, such as program modules comprising executable program instructions, being executable by the computer device 10. Generally, program modules may include routines, programs, objects, components, logic, data structures, and so on that perform particular tasks or implement particular abstract data types. Computer device 10 may be practiced in distributed computing environments where tasks are performed by remote processing devices that are linked through a communications network. In a distributed computing environment, program modules may be located in both local and remote computer device storage media including memory storage devices.

In FIG. 13, computer device 10 is shown in the form of an exemplary general-purpose computing device. The components of computer device 10 may include, but are not limited to, one or more processors or processing units 16, a system memory 28, and a bus 18 that couples various system components including system memory 28 to processor 16. Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer device 10 may comprise a variety of computer device readable storage media. Such media may be any available storage media accessible by computer device 10, and include both volatile and non-volatile storage media, removable and non-removable storage media.

A system memory 28 may include computer device readable storage media in the form of volatile memory, such as random-access memory (RAM) 30 and/or cache memory 32. Computer device 10 may further include other removable/non-removable, volatile/non-volatile computer device storage media. For example, storage system 34 may be provided for reading from and writing to a non-removable, non-volatile magnetic media also referred to as a hard drive. For example, a magnetic disk drive for reading from and writing to a removable, non-volatile magnetic disk, e.g., a floppy disk, and an optical disk drive for reading from or writing to a removable, non-volatile optical disk such as a CD-ROM, DVD-ROM or other optical storage media may be provided. In such instances, each storage medium may be connected to bus 18 by one or more data media interfaces. Memory 28 may, e.g., include a three-dimensional digital tissue model of intraoral tissue of the patient's oral cavity comprising maxillary and mandibular tissue. The three-dimensional digital tissue model of intraoral tissue may, e.g., comprise scan data of the intraoral tissue. The scan data of the intraoral tissue may, e.g., comprise scan data acquired using a medical imaging technique, like computed tomography (CT), cone beam computed tomography (CBCT), and/or digital volume tomography (DVT). The scan data of the intraoral tissue may comprise, e.g., optical scan data. The optical scan data may, e.g., comprise intraoral optical scan data or optical scan data from an optical scan of a classical mold/impression of the intraoral tissue. The optical scan data may, e.g., provide information about the surface structure of the patient's intraoral tissue comprising teeth and the gingiva.

Memory 28 may, e.g., include a maximum mouth opening value descriptive of a maximum degree of mouth opening achievable by the patient. For example, the maximum mouth opening value is a value determined using a maximal interincisal opening (MIO) measurement or a range of motion (ROM) measurement.

Memory 28 may, e.g., include a definition of a three-dimensional working space required for arranging and using one or more selected treatment elements for executing the intraoral treatment. The three-dimensional working space may be defined using size information of the one or more selected treatment elements.

Memory 28 may, e.g., include size information of one or more selected treatment elements. For example, memory 28 may comprise one or more three-dimensional digital models of the one or more selected treatment elements comprising the size information of the respective one or more selected treatment elements.

Memory 28 may, e.g., include a clearance value defining a size of an additional clearance required for a moving and positioning one or more selected treatment elements at a test position with the test orientation, the clearance value being used for determining the three-dimensional working space.

Memory 28 may, e.g., include a digital articulator configured for configured to simulate movements of the mandible in relation to the maxilla, e.g., an opening of the patient's mouth until the maximum degree of mouth opening described by the maximum mouth opening value is achieved.

Memory 28 may, e.g., include jaw motion tracking data, i.e., tracking information of a tracking of jaw movements of the patient's, which may be used for defining a movement of the maxillary and mandibular tissue relative to each other with the digital articulator.

Memory 28 may, e.g., include averaged moving information of human jaw movements, which may be used for defining a movement of the maxillary and mandibular tissue relative to each other with the digital articulator.

Memory 28 may, e.g., include for controlling a manufacturing of one or more treatment elements to be manufactured. The data defines one or more three-dimensional digital models of one or more treatment elements to be manufactured as templates for the one or more treatment elements to be manufactured. The data for controlling a manufacturing of the one or more treatment elements may be provided using the computer device 10.

Program 40 may have a set of one or more program modules 42 and by way of example be stored in memory 28. The program modules 42 may comprise an operating system, one or more application programs, other program modules, and/or program data. Each of these program modules 42, i.e., the operating system, the one or more application programs, the other program modules, and/or the program data or some combination thereof, may include an implementation of a networking environment. One or more of the program modules 42 may be configured for executing a method for planning an intraoral treatment for a patient. One or more of the program modules 42 may be configured for controlling a manufacturing of one or more treatment elements. One or more of the program modules 42 may, e.g., be configured for executing one or more of the methods of FIG. 9 to 12.

Computer device 10 may further communicate with one or more external devices 14 such as a keyboard, a pointing device, like a mouse, and a display 24 enabling a user to interact with computer device 10. Such communication can occur via input/output (I/O) interfaces 22. Computer device 10 may further communicate with one or more networks such as a local area network (LAN), a general wide area network (WAN), and/or a public network, like the Internet, via network adapter 20. Network adapter 20 may communicate with other components of computer device 10 via bus 18. It should be understood that although not shown, other hardware and/or software components could be used in conjunction with computer device 10.

The computer device 10 shown in FIG. 13 may be configured for planning an intraoral treatment for a patient. The method comprises receiving a three-dimensional digital tissue model of intraoral tissue of the patient's oral cavity comprising maxillary and mandibular tissue. Further, a maximum mouth opening value descriptive of a maximum degree of mouth opening achievable by the patient is received. Relative positions of the maxillary and mandibular tissue of the three-dimensional digital tissue model relative to each other are determined for a maximum mouth opening of the patient using the maximum mouth opening value. Size information descriptive of sizes of one or more treatment elements selected to be used for executing the intraoral treatment are received. A three-dimensional working space required for arranging and using the one or more selected treatment elements for executing the intraoral treatment is determined using the size information of the one or more selected treatment elements.

For a test position and test orientation of the one or more selected treatment elements within the three-dimensional digital tissue model with the maxillary and mandibular tissue arranged at the determined relative positions it is checked, whether the working space arranged at the test position and aligned with the test orientation intersects with tissue of the three-dimensional digital tissue model opposite of the test position. In response to a detection of an intersection, an indication signal indicating the detected intersection is output and a planning adjustment for the planning of the intraoral treatment configured to prevent the detected intersection is determined.

The computer device 10 shown in FIG. 13 may, e.g., further be configured for controlling a manufacturing of one or more treatment elements using data provided for controlling the manufacturing. The manufactured treatment elements may be physical copies of templates defined by the provided data.

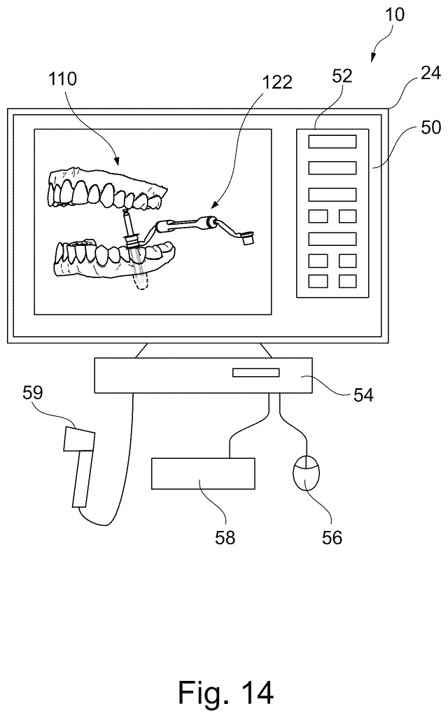

FIG. 14 shows an exemplary computer device 10 for planning an intraoral treatment for a patient. The computer device 10 may, e.g., be configured as shown in FIG. 13. The computer device 10 may comprise a hardware component 54 comprising one or more processors as well as a memory storing machine-executable program instructions. Execution of the program instructions by the one or more processors may cause the one or more processors to control the computer device 10 to, e.g., plan planning an intraoral treatment for a patient. In response to a detection of an intersection, the computer device 10 may be configured to output an indication signal indicating the detected intersection as well as to determine a planning adjustment for the planning of the intraoral treatment configured to prevent the detected intersection.

The computer device 10 may further comprise one or more input devices, like a keyboard 58 and a mouse 56, enabling a user to interact with the computer device 10. Furthermore, the computer device 10 may comprise one or more output devices, like a display 24 providing a graphical user interface 50 with control elements 52, e.g., GUI elements, enabling the user to control the planning of the intraoral treatment for the patient. The computer device 10 may further comprise an exemplary scanner 59 configured for scanning a patient's mouth. The scanner 59 may, e.g., comprise an CT or CBCT scanning system. The scanner 59 may, e.g., comprise an optical scanner configured for scanning, e.g., a patient's oral cavity, an imprint of a patient's oral cavity and/or a positive of a patient's oral cavity generated using an imprint.

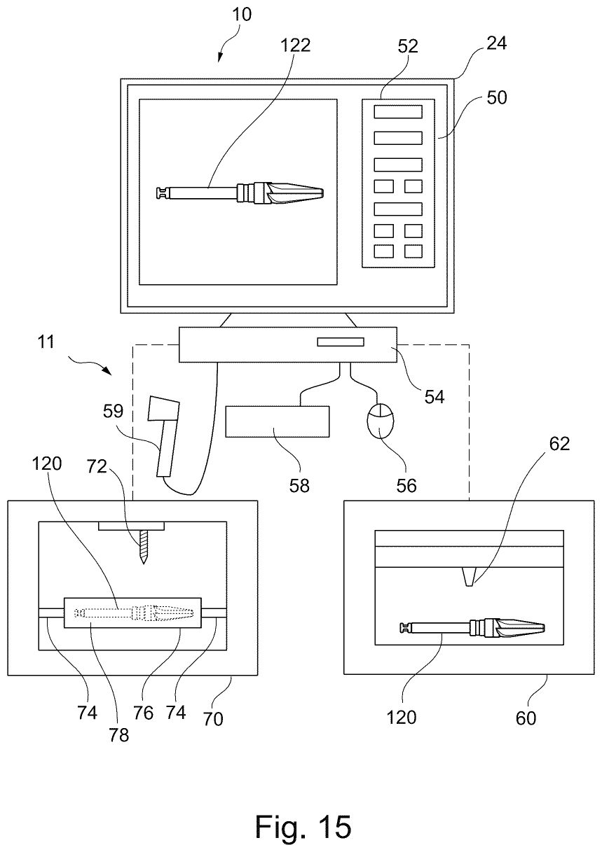

FIG. 15 shows an exemplary manufacturing system 11 for manufacturing one or more treatment elements 120. The one or more treatment elements 120 being manufactured may, e.g., comprise one or more treatment tools and/or one or more dental elements. For manufacturing the one or more treatment elements 120, data may be used provided for controlling the manufacturing. The data provided for controlling the manufacturing of the one or more treatment elements 120 may define one or more three-dimensional digital models 122 of the treatment elements 120 to be manufactures as templates for the treatment elements 120. The one or more manufactured treatment elements 120 may be physical copies of the templates defined by the provided data.

The manufacturing system 11 may comprise the computer device 10 of FIG. 14. The computer device 10 may further be configured to control one or more manufacturing devices 60, 70. For controlling the one or more manufacturing devices 60, 70 a processing unit of the computer device 10 may execute program instructions. Execution of the program instructions by the processing unit may cause the computer device 10 to control the one or more manufacturing devices 60, 70 to manufacture the one or more treatment elements 120 using the data provided for controlling the manufacturing.

For example, the manufacturing system 11 may comprise a manufacturing device in form of a machining device 70 controlled by the computer device 10. The machining device 70 may be configured to machining a blank 76 using one or more machining tools 72. The blank 76 of raw material 78, may be provided using one or more holding devices 74 and cut into a desired shape and size of the element to be manufactured, e.g., a treatment element 120. The machining tool 72 may, e.g., be a milling tool. In particular for manufacturing treatment tools, the raw material 78 may, e.g., be a metal or a metal alloy.

For example, the manufacturing system 11 may comprise a manufacturing device in form of a three-dimensional (3D) printing device 60. The 3D printing device 60 may be controlled by the computer device 10 and configured to print an element to be manufactured, e.g., a treatment element 120. The 3D printing device 60 may comprise a printing element 62 configured to generate the respective element, like the treatment element 120, layer by layer. The printing element 62 may, e.g., comprise a nozzle configured for distributing printing material.

In case the element to be manufactured using the 3D printing device 60 is made using metal, like a treatment tool, the 3D printing device 60 may, e.g., be configured for executing selective laser sintering, laser melting or electron beam melting. Selective laser sintering uses a laser for sintering a powdered material, aiming the laser automatically at points in space defined by a three-dimensional digital model of the element to be printed. The laser energy may result in a local sintering of the powdered material, binding the material together to create a solid structure. For example, the printing element 62 of the 3D printing device 60 may comprise a laser and/or a distributing device for distributing the powdered material. In case of laser melting, the laser is used for locally melting the powdered material, binding the material together to create a solid structure. Selective electron beam melting uses an electron beam for melting a powdered material, aiming the electron beam automatically at points in space defined by a three-dimensional digital model of the element to be printed. The beam energy may result in a local melting of the powdered material, binding the material together to create a solid structure. For example, the printing element 62 of the 3D printing device 60 may comprise an electron beam emitter and/or a distributing device for distributing the powdered material.

For example, the three-dimensional digital model 122 may be used as a positive to define a negative of the treatment element 120 in form of a negative three-dimensional model. The negative three-dimensional digital model may be used to manufacture, e.g., using machining device 70 or 3D printing device 60, a casting matrix. The casting matrix may be configured for casting the treatment element 120 by inserting casting material into the casting matrix and curing the inserted casting material.

The one or more treatment elements 120 being manufactured may, e.g., comprise one or more dental elements. For example, the one or more dental elements being manufactured comprise one or more of the following: an implant, an abutment, a screw, a crown, a bridge.

The one or more treatment elements 120 being manufactured may, e.g., comprise one or more treatment tools. For example, the one or more treatment tools being manufactured comprise one or more of the following: an implant drill, an implant drill handle, a drill guide handle, a sleeve, a surgery guide, an implant contra-angle.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

A single processor or other unit may fulfill the functions of several items recited in the claims. A computer program may be stored/distributed on a suitable medium, such as an optical storage medium or a solid-state medium supplied together with or as part of other hardware, but may also be distributed in other forms, such as via the Internet or other wired or wireless telecommunication systems.

As will be appreciated by one skilled in the art, aspects of the present invention may be embodied as an apparatus, method, computer program or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon. A computer program comprises the computer executable code or “program instructions”.

Any combination of one or more computer readable medium(s) may be utilized. The computer readable medium may be a computer readable signal medium or a computer readable storage medium. A “computer-readable storage medium” as used herein encompasses any tangible storage medium which may store instructions which are executable by a processor of a computing device, also referred to as a processing unit. The computer-readable storage medium may be referred to as a computer-readable non-transitory storage medium. The computer-readable storage medium may also be referred to as a tangible computer readable medium. For example, a computer-readable storage medium may also be able to store data which is able to be accessed by the processor of the computing device. Examples of computer-readable storage media include, but are not limited to: a floppy disk, a magnetic hard disk drive, a solid-state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. A further example of an optical disk may be a Blu-ray disk. The term computer readable-storage medium also refers to various types of recording media capable of being accessed by the computer device via a network or communication link. For example, a data may be retrieved over a modem, over the internet, or over a local area network. Computer executable code embodied on a computer readable medium may be transmitted using any appropriate medium, including but not limited to wireless, wireline, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

“Computer memory” or “memory” is an example of a computer-readable storage medium. Computer memory is any memory which is directly accessible to a processor. “Computer storage” or “storage” is a further example of a computer-readable storage medium. Computer storage is any non-volatile computer-readable storage medium. For example, computer storage may also be computer memory or vice versa.

A “processor” or “processing unit” as used herein encompasses an electronic component which is able to execute a program or machine executable instruction or computer executable code. References to the computing device comprising “a processor” should be interpreted as possibly containing more than one processor or processing core. The processor may for instance be a multi-core processor. A processor may also refer to a collection of processors within a single computer device or distributed amongst multiple computer devices. The term computing device should also be interpreted to possibly refer to a collection or network of computing devices each comprising a processor or processors. The computer executable code may be executed by multiple processors that may be within the same computing device or which may even be distributed across multiple computing devices.

Computer executable code may comprise machine executable instructions or a program which causes a processor to perform an aspect of the present invention. Computer executable code for carrying out operations for aspects of the present invention may be written in any combination of one or more programming languages, including an object-oriented programming language such as Java, Smalltalk, C++ or the like and conventional procedural programming languages, such as the “C” programming language or similar programming languages and compiled into machine executable instructions. In some instances, the computer executable code may be in the form of a high-level language or in a pre-compiled form and be used in conjunction with an interpreter which generates the machine executable instructions on the fly.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Generally, the program instructions can be executed on one processor or on several processors. In the case of multiple processors, they can be distributed over several different entities like clients, servers etc. Each processor could execute a portion of the instructions intended for that entity. Thus, when referring to a system or process involving multiple entities, the computer program or program instructions are understood to be adapted to be executed by a processor associated or related to the respective entity.

A “user interface” as used herein is an interface which allows a user or operator to interact with a computer or computer device. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer to indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, gear sticks, steering wheel, pedals, wired glove, dance pad, remote control, one or more switches, one or more buttons, and accelerometer are all examples of user interface components which enable the receiving of information or data from an operator.

A GUI element is a data object some of which's attributes specify the shape, layout and/or behavior of an area displayed on a graphical user interface, e.g., a screen. A GUI element can be a standard GUI element such as a button, a text box, a tab, an icon, a text field, a pane, a check-box item or item group or the like. A GUI element can likewise be an image, an alphanumeric character or any combination thereof. At least some of the properties of the displayed GUI elements depend on the data value aggregated on the group of data object said GUI element represents.

Aspects of the present invention are described with reference to flowchart illustrations and/or block diagrams of methods, apparatus (systems) and computer program products. It will be understood that each block or a portion of the blocks of the flowchart, illustrations, and/or block diagrams, can be implemented by computer program instructions in form of computer executable code when applicable. It is further understood that, when not mutually exclusive, combinations of blocks in different flowcharts, illustrations, and/or block diagrams may be combined. These computer program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks.

Although the invention may have been described in reference to specific examples, it should be understood that the invention is not limited to these examples only and that many variations of these examples may be readily envisioned by the skilled person after having read the present disclosure. The invention may thus further be described without limitation and by way of example only by the following embodiments. The following embodiments may contain preferred embodiments. Accordingly, the term “feature combination” as used therein may refer to such a “preferred embodiment”.
1. A computer-implemented method for planning an intraoral treatment for a patient,
   the method comprising:
   receiving a three-dimensional digital tissue model of intraoral tissue of the patient's oral cavity comprising maxillary and mandibular tissue,
   receiving a maximum mouth opening value descriptive of a maximum degree of mouth opening achievable by the patient,
   determining relative positions of the maxillary and mandibular tissue of the three-dimensional digital tissue model relative to each other for a maximum mouth opening of the patient using the maximum mouth opening value,
   receiving size information descriptive of sizes of one or more treatment elements selected to be used for executing the intraoral treatment,
   determining a three-dimensional working space required for arranging and using the one or more selected treatment elements for executing the intraoral treatment using the size information of the one or more selected treatment elements,
   checking for a test position and test orientation of the one or more selected treatment elements within the three-dimensional digital tissue model with the maxillary and mandibular tissue arranged at the determined relative positions, whether the working space arranged at the test position and aligned with the test orientation intersects with tissue of the three-dimensional digital tissue model opposite of the test position,
   in response to a detection of an intersection, outputting an indication signal indicating the detected intersection and determining a planning adjustment for the planning of the intraoral treatment configured to prevent the detected intersection.
2. The method of feature combination 1, the method further comprising:
   receiving a clearance value defining a size of an additional clearance required for a moving and positioning of the one or more selected treatment elements at the test position with the test orientation, the clearance value being used for determining the three-dimensional working space.
3. The method of any of the preceding feature combinations, the method further comprising using a digital articulator for determining the relative positions of the maxillary and mandibular tissue relative to each other.
4. The method of feature combination 3, for the determining of the relative positions of the maxillary and mandibular tissue relative to each other using the digital articulator, the method further comprising:
   receiving tracking information of a tracking of jaw movements of the patient's and using the received tracking information for defining a movement of the maxillary and mandibular tissue relative to each other with the digital articulator, or
   receiving averaged moving information of human jaw movements and using the received moving information for defining the movement of the maxillary and mandibular tissue relative to each other with the digital articulator.
5. The method of any of the previous feature combinations, the size information descriptive of the sizes of the one or more selected treatment elements being received from one or more libraries of treatment elements comprising a plurality of entries assigned to different treatment elements of a plurality of different treatment elements with individual entries of the plurality of entries comprising size information of individual treatment elements of the plurality of treatment elements, to which the individual entries are assigned.
6. The method of any of the previous feature combinations, the outputting of the indication signal comprising a highlighting of the intersection in a graphical visualization of the three-dimensional digital tissue model with the maxillary and mandibular tissue arranged in the relative positions relative to each other, wherein the graphical visualization of the three-dimensional digital tissue model with the highlighted intersection is output using a graphical user interface.
7. The method of any of the previous feature combinations, the method further comprising providing the planning adjustment and preventing the detected intersection.
8. The method of any of the previous feature combinations, the test position for the one or more selected treatment elements being a position of a plurality of potential position tested for the planned treatment, the determined planning adjustment compromising a restriction of a selectability of the positions of the plurality of position with the test position being excluded from the selectability for the planned treatment.
9. The method of any of the previous feature combinations, the test orientation of the one or more selected treatment elements being an orientation of a plurality of potential orientation tested for the planned treatment, the determined planning adjustment compromising a restriction of a selectability of the orientations of the plurality of orientation with the test orientation being excluded from the selectability for the planned treatment.
10. The method of any of the previous feature combinations, the method further comprising outputting a graphical visualization of the treatment elements using the graphical user interface, the determined planning adjustment comprising a restriction of a movability of the treatment elements relative to the three-dimensional digital tissue model, wherein one or more of the following is excluded: a moving of the treatment elements to the test position, a moving of the treatment elements into alignment with the test orientation.
11. The method of any of the previous feature combinations, the determining of the planning adjustment comprising:
   determining an adjusted position as a replacement for the test position, at which an intersection of the three-dimensional workspace with tissue of the three-dimensional digital tissue model opposite of the adjusted position is prevented,
   outputting the adjusted position.
12. The method of feature combination 11, the outputting of the adjusted position comprising a highlighting of the adjusted position in the graphical visualization of the three-dimensional digital tissue model with the maxillary and mandibular tissue arranged in the relative positions relative to each other using the graphical user interface.
13. The method of any of the previous feature combinations, the determining of the planning adjustment comprising:
   determining an adjusted orientation as a replacement for the test orientation, for which an intersection of the three-dimensional workspace with tissue of the three-dimensional digital tissue model opposite of the test orientation is prevented,
   outputting the adjusted orientation.
14. The method of feature combination 13, the outputting of the adjusted orientation comprising a highlighting of the adjusted orientation in the graphical visualization of the three-dimensional digital tissue model with the maxillary and mandibular tissue arranged in the relative orientations relative to each other using the graphical user interface.
15. The method of any of feature combination 1 to 10, the determining of the planning adjustment comprising:
   determining one or more replacement treatment elements as replacements for one or more of the planned treatment elements, the replacement treatment elements having sizes resulting in an adjusted three-dimensional workspace, for which at the test position with the test orientation an intersection with tissue of the three-dimensional digital tissue model opposite of the test orientation is prevented,
   outputting an identification signal identifying the one or more replacement treatment elements.
16. The method of feature combination 15, the outputting of the one or more replacement treatment elements comprising outputting a graphical visualization of the one or more replacement treatment elements within the three-dimensional digital tissue model with the maxillary and mandibular tissue arranged in the relative positions relative to each other using the graphical user interface, wherein the one or more replacement treatment elements are arranged at the test position and aligned with the test orientation.
17. The method of any of the previous feature combinations, the one or more selected treatment elements comprising one or more treatment tools.
18. The method of any of the previous feature combinations, the one or more selected treatment elements comprising one or more dental elements.
19. A computer program for planning of an intraoral treatment for a patient, the computer program comprising program instructions executable by a processor of a computer device to cause the computer device to:
   receive a three-dimensional digital tissue model of intraoral tissue of the patient's oral cavity comprising maxillary and mandibular tissue,
   receive a maximum mouth opening value descriptive of a maximum degree of mouth opening achievable by the patient,
   determine relative positions of the maxillary and mandibular tissue of the three-dimensional digital tissue model relative to each other for a maximum mouth opening of the patient using the maximum mouth opening value,
   receive size information descriptive of sizes of one or more treatment elements selected to be used for executing the intraoral treatment,
   determine a three-dimensional working space required for arranging and using the one or more selected treatment elements for executing the intraoral treatment using the size information of the one or more selected treatment elements,
   check for a test position and test orientation of the one or more selected treatment elements within the three-dimensional digital tissue model with the maxillary and mandibular tissue arranged at the determined relative positions, whether the working space arranged at the test position and aligned with the test orientation intersects with tissue of the three-dimensional digital tissue model opposite of the test position,
   in response to a detection of an intersection, output an indication signal indicating the detected intersection and determining a planning adjustment for the planning of the intraoral treatment configured to prevent the detected intersection.
20. A computer program product for planning of an intraoral treatment for a patient, the computer program product comprising a non-transitory computer readable storage medium having program instructions embodied therewith, the program instructions being executable by a processor of a computer device to cause the computer device to:
   receive a three-dimensional digital tissue model of intraoral tissue of the patient's oral cavity comprising maxillary and mandibular tissue,
   receive a maximum mouth opening value descriptive of a maximum degree of mouth opening achievable by the patient,
   determine relative positions of the maxillary and mandibular tissue of the three-dimensional digital tissue model relative to each other for a maximum mouth opening of the patient using the maximum mouth opening value,
   receive size information descriptive of sizes of one or more treatment elements selected to be used for executing the intraoral treatment,
   determine a three-dimensional working space required for arranging and using the one or more selected treatment elements for executing the intraoral treatment using the size information of the one or more selected treatment elements,
   check for a test position and test orientation of the one or more selected treatment elements within the three-dimensional digital tissue model with the maxillary and mandibular tissue arranged at the determined relative positions, whether the working space arranged at the test position and aligned with the test orientation intersects with tissue of the three-dimensional digital tissue model opposite of the test position,
   in response to a detection of an intersection, output an indication signal indicating the detected intersection and determining a planning adjustment for the planning of the intraoral treatment configured to prevent the detected intersection.
21. A computer device for planning of an intraoral treatment for a patient, the computer device comprising a processor and a memory storing program instructions executable by the processor, execution of the program instructions by the processor causing the computer device to:
   receive a three-dimensional digital tissue model of intraoral tissue of the patient's oral cavity comprising maxillary and mandibular tissue,
   receive a maximum mouth opening value descriptive of a maximum degree of mouth opening achievable by the patient,
   determine relative positions of the maxillary and mandibular tissue of the three-dimensional digital tissue model relative to each other for a maximum mouth opening of the patient using the maximum mouth opening value,
   receive size information descriptive of sizes of one or more treatment elements selected to be used for executing the intraoral treatment,
   determine a three-dimensional working space required for arranging and using the one or more selected treatment elements for executing the intraoral treatment using the size information of the one or more selected treatment elements,
   check for a test position and test orientation of the one or more selected treatment elements within the three-dimensional digital tissue model with the maxillary and mandibular tissue arranged at the determined relative positions, whether the working space arranged at the test position and aligned with the test orientation intersects with tissue of the three-dimensional digital tissue model opposite of the test position,
   in response to a detection of an intersection, output an indication signal indicating the detected intersection and determining a planning adjustment for the planning of the intraoral treatment configured to prevent the detected intersection.

REFERENCE SIGNS LIST

| | |
|---|---|
| 10 | computer device |
| 11 | manufacturing system |
| 14 | external device |
| 16 | processing unit |
| 18 | bus |
| 20 | network adapter |
| 22 | I/O interface |
| 24 | display |
| 28 | memory |
| 30 | RAM |
| 32 | cache |
| 34 | storage system |
| 40 | program |
| 42 | program module |
| 50 | user interface |
| 52 | control elements |
| 54 | hardware device |
| 56 | keyboard |
| 58 | mouse |
| 59 | scanner |
| 60 | 3D printing device |
| 62 | printing element |
| 70 | machining device |
| 72 | machining tool |
| 74 | holding device |
| 76 | blank |
| 78 | raw material |
| 100 | intraoral tissue |
| 102 | maxilla |
| 103 | central maxillary incisor |
| 104 | mandible |
| 105 | central mandibular incisor |
| 110 | 3D digital tissue model |
| 112 | maxillary tissue |
| 114 | mandibular tissue |
| 120 | treatment element |
| 121 | pilot drill |
| 122 | 3D digital model of treatment |
| 123 | tapered drill |
| 124 | implant |
| 125 | threaded tapered drill |
| 126 | drill guide handle |
| 127 | through hole |
| 130 | drilling hole |
| 140 | 3D working space |
| 144 | clearance |
| 142 | 3D working space |
| 150 | test position |
| 152 | test orientation |
| 154 | region |
| 155 | radius |
| 156 | cone |
| 158 | axis |
| 159 | generatrix |
| 160 | scale |
| 170 | digital articulator |
| 172 | upper articulator part |
| 174 | lower articulator part |
| 176 | hinge |
| θ | angle |

1. A computer-implemented method for planning an intraoral treatment for a patient,
the method comprising:
receiving a three-dimensional digital tissue model of intraoral tissue of the patient's oral cavity comprising maxillary and mandibular tissue,
receiving a maximum mouth opening value descriptive of a maximum degree of mouth opening achievable by the patient,
determining relative positions of the maxillary and mandibular tissue of the three-dimensional digital tissue model relative to each other for a maximum mouth opening of the patient using the maximum mouth opening value,
receiving size information descriptive of sizes of one or more treatment elements selected to be used for executing the intraoral treatment,
determining a three-dimensional working space required for arranging and using the one or more selected treatment elements for executing the intraoral treatment using the size information of the one or more selected treatment elements,
checking for a test position and test orientation of the one or more selected treatment elements within the three-dimensional digital tissue model with the maxillary and mandibular tissue arranged at the determined relative positions, whether the working space arranged at the test position and aligned with the test orientation intersects with tissue of the three-dimensional digital tissue model opposite of the test position,
in response to a detection of an intersection, outputting an indication signal indicating the detected intersection and determining a planning adjustment for the planning of the intraoral treatment configured to prevent the detected intersection.

2. The method of claim 1, the method further comprising:
receiving a clearance value defining a size of an additional clearance required for a moving and positioning of the one or more selected treatment elements at the test position with the test orientation, the clearance value being used for determining the three-dimensional working space.

3. The method of claim 1, the method further comprising using a digital articulator for determining the relative positions of the maxillary and mandibular tissue relative to each other.

4. The method of claim 3, for the determining of the relative positions of the maxillary and mandibular tissue relative to each other using the digital articulator, the method further comprising:
receiving tracking information of a tracking of jaw movements of the patient's and using the received tracking information for defining a movement of the maxillary and mandibular tissue relative to each other with the digital articulator, or
receiving averaged moving information of human jaw movements and using the received moving information for defining the movement of the maxillary and mandibular tissue relative to each other with the digital articulator.

5. The method of claim 1, the size information descriptive of the sizes of the one or more selected treatment elements being received from one or more libraries of treatment elements comprising a plurality of entries assigned to different treatment elements of a plurality of different treatment elements with individual entries of the plurality of entries comprising size information of individual treatment elements of the plurality of treatment elements, to which the individual entries are assigned.

6. The method of claim 1, the outputting of the indication signal comprising a highlighting of the intersection in a graphical visualization of the three-dimensional digital tissue model with the maxillary and mandibular tissue arranged in the relative positions relative to each other, wherein the graphical visualization of the three-dimensional digital tissue model with the highlighted intersection is output using a graphical user interface.

7. The method of claim 1, the method further comprising providing the planning adjustment and preventing the detected intersection.

8. The method of claim 1, the test position for the one or more selected treatment elements being a position of a plurality of potential position tested for the planned treatment, the determined planning adjustment compromising a restriction of a selectability of the positions of the plurality of position with the test position being excluded from the selectability for the planned treatment.

9. The method of claim 1, the test orientation of the one or more selected treatment elements being an orientation of a plurality of potential orientation tested for the planned treatment, the determined planning adjustment compromising a restriction of a selectability of the orientations of the plurality of orientation with the test orientation being excluded from the selectability for the planned treatment.

10. The method of claim 1, the method further comprising outputting a graphical visualization of the treatment elements using the graphical user interface, the determined planning adjustment comprising a restriction of a movability of the treatment elements relative to the three-dimensional digital tissue model, wherein one or more of the following is excluded: a moving of the treatment elements to the test position, a moving of the treatment elements into alignment with the test orientation.

11. The method of claim 1, the determining of the planning adjustment comprising:
determining an adjusted position as a replacement for the test position, at which an intersection of the three-dimensional workspace with tissue of the three-dimensional digital tissue model opposite of the adjusted position is prevented,
outputting the adjusted position.

12. The method of claim 11, the outputting of the adjusted position comprising a highlighting of the adjusted position in the graphical visualization of the three-dimensional digital tissue model with the maxillary and mandibular tissue arranged in the relative positions relative to each other using the graphical user interface.

13. The method of claim 1, the determining of the planning adjustment comprising:
determining an adjusted orientation as a replacement for the test orientation, for which an intersection of the three-dimensional workspace with tissue of the three-dimensional digital tissue model opposite of the test orientation is prevented,
outputting the adjusted orientation.

14. The method of claim 13, the outputting of the adjusted orientation comprising a highlighting of the adjusted orientation in the graphical visualization of the three-dimensional digital tissue model with the maxillary and mandibular tissue arranged in the relative orientations relative to each other using the graphical user interface.

15. The method of claim 1, the determining of the planning adjustment comprising:
determining one or more replacement treatment elements as replacements for one or more of the planned treatment elements, the replacement treatment elements having sizes resulting in an adjusted three-dimensional workspace, for which at the test position with the test orientation an intersection with tissue of the three-dimensional digital tissue model opposite of the test orientation is prevented,
outputting an identification signal identifying the one or more replacement treatment elements.

16. The method of claim 15, the outputting of the one or more replacement treatment elements comprising outputting a graphical visualization of the one or more replacement treatment elements within the three-dimensional digital tissue model with the maxillary and mandibular tissue arranged in the relative positions relative to each other using the graphical user interface, wherein the one or more replacement treatment elements are arranged at the test position and aligned with the test orientation.

17. The method of claim 1, the one or more selected treatment elements comprising one or more treatment tools.

18. The method of claim 1, the one or more selected treatment elements comprising one or more dental elements.

19. A computer program product for planning of an intraoral treatment for a patient, the computer program product comprising a non-transitory computer readable storage medium having program instructions embodied therewith, the program instructions being executable by a processor of a computer device to cause the computer device to:
receive a three-dimensional digital tissue model of intraoral tissue of the patient's oral cavity comprising maxillary and mandibular tissue,
receive a maximum mouth opening value descriptive of a maximum degree of mouth opening achievable by the patient,
determine relative positions of the maxillary and mandibular tissue of the three-dimensional digital tissue model relative to each other for a maximum mouth opening of the patient using the maximum mouth opening value,
receive size information descriptive of sizes of one or more treatment elements selected to be used for executing the intraoral treatment,
determine a three-dimensional working space required for arranging and using the one or more selected treatment elements for executing the intraoral treatment using the size information of the one or more selected treatment elements,
check for a test position and test orientation of the one or more selected treatment elements within the three-dimensional digital tissue model with the maxillary and mandibular tissue arranged at the determined relative positions, whether the working space arranged at the test position and aligned with the test orientation intersects with tissue of the three-dimensional digital tissue model opposite of the test position,
in response to a detection of an intersection, output an indication signal indicating the detected intersection and determining a planning adjustment for the planning of the intraoral treatment configured to prevent the detected intersection.

20. A computer device for planning of an intraoral treatment for a patient, the computer device comprising a processor and a memory storing program instructions executable by the processor, execution of the program instructions by the processor causing the computer device to:
receive a three-dimensional digital tissue model of intraoral tissue of the patient's oral cavity comprising maxillary and mandibular tissue,
receive a maximum mouth opening value descriptive of a maximum degree of mouth opening achievable by the patient,
determine relative positions of the maxillary and mandibular tissue of the three-dimensional digital tissue model relative to each other for a maximum mouth opening of the patient using the maximum mouth opening value,
receive size information descriptive of sizes of one or more treatment elements selected to be used for executing the intraoral treatment,
determine a three-dimensional working space required for arranging and using the one or more selected treatment elements for executing the intraoral treatment using the size information of the one or more selected treatment elements,
check for a test position and test orientation of the one or more selected treatment elements within the three-dimensional digital tissue model with the maxillary and mandibular tissue arranged at the determined relative positions, whether the working space arranged at the test position and aligned with the test orientation intersects with tissue of the three-dimensional digital tissue model opposite of the test position,
in response to a detection of an intersection, output an indication signal indicating the detected intersection and determining a planning adjustment for the planning of the intraoral treatment configured to prevent the detected intersection.
